(19) United States
(12) Patent Application Publication
(10) Pub. No.: US 2011/0245929 A1
(43) Pub. Date: Oct. 6, 2011

(54) METHODS AND COMPOSITIONS FOR JOINT HEALING AND REPAIR

(75) Inventors: Kevin Rakin, Westport, CT (US); Charles E. Hart, Brentwood, TN (US); Conan Young, Nashville, TN (US); Ronda Schreiber, Poway, CA (US)

(73) Assignee: Advanced BioHealing Inc., La Jolla, CA (US)

(21) Appl. No.: 13/042,295

(22) Filed: Mar. 7, 2011

Related U.S. Application Data
(60) Provisional application No. 61/311,270, filed on Mar. 5, 2010.
(60) Provisional application No. 61/366,097, filed on Jul. 20, 2010.

(51) Int. Cl. A61F 2/02; A61B 17/04; A61B 17/32

(52) U.S. Cl. 623/23.72; 606/228; 606/167

(57) ABSTRACT

The invention in some aspects provides implantable articles comprising in vitro-prepared tissues for joint repair. Devices and methods for introducing implantable articles into a subject are also provided. In some aspects of the invention devices and systems for minimally invasive surgery are provided. In some aspects, methods are provided for regenerating a bone-tendon interface in a subject by implanting an in vitro-prepared tissue between a detached tendon or detached ligament and a bone in a subject. In other aspects, methods are provided for maintaining exogenous, viable fibroblasts between a detached tendon and a bone in a subject. In other aspects, methods are provided for delivering exogenous cytokines and/or growth factors to a damaged bone-tendon interface.

### RELATED APPLICATIONS

This application claims priority under 35 U.S.C. §119(e) from U.S. provisional application Ser. No. 61/311,270, filed Mar. 5, 2010 and U.S. provisional application Ser. No. 61/366,097, filed Jul. 20, 2010, the contents of each of which are incorporated by reference herein in their entireties.

### FIELD OF THE INVENTION

The invention relates to methods and compositions for joint repair.

### BACKGROUND OF INVENTION

A variety of different treatments exist for joint repair ranging from simple rest, physical therapy, and anti-inflammatory medications to invasive surgical procedures. Many joint injuries are repaired surgically using an arthroscopic approach. This can involve the insertion of several cannulae by the surgeon into the tissues to be repaired, often one for delivery of sutures and hardware, and others fitted with cameras to allow visualization of the repair site internally. The procedures are typically conducted with irrigation of the site to provide a better view of the surgery. The decision to select one or another treatment method depends on a variety of parameters, such as the age, metabolic status and activity level of the subject and the nature and severity of the damage. Existing treatments often suffer from a high rate of re-injury. Thus, the need exists for improved methods for treating joint damage.

### SUMMARY OF INVENTION

Methods are provided herein for joint repair, including repairing tendon or ligament damage. According to some aspects of the invention, it has been discovered that an in vitro-prepared tissue, implanted according to the methods disclosed herein, can effectively stimulate regeneration of bone-tendon interfaces and re-establish tendon function.

The invention in some aspects relates to implantable articles comprising in vitro-prepared tissues for joint repair. Implantable articles of the invention are suited for implantation between a detached tendon or ligament and bone. In some embodiments, the implantable articles are designed to slide onto a suture connecting a detached tendon or ligament and bone. The implantable articles are well suited for delivery using minimally invasive techniques as well as delivery when open surgical approaches are utilized. Accordingly, in some aspects of the invention, devices and systems for minimally invasive surgery are provided. Implantable articles delivered according to the methods disclosed herein, can effectively stimulate regeneration of bone-tendon interfaces and bone-ligament interfaces and re-establish joint function. In some embodiments, the in vitro-prepared tissue comprises fibroblasts grown on a scaffold where the fibroblasts replicate and produce natural proteins such as growth factors, cytokines, collagens, and glycosaminoglycans. In some embodiments, the scaffold may be synthetic in origin or may be composed of natural tissues, where the scaffold is absorbed by the subject while the subject maintains, and does not reject, the in vitro-prepared tissue. In some embodiments, the fibroblasts remain at the implantation site after the scaffold is absorbed. Thus, in some aspects, methods are provided for regenerating bone-tendon and bone-ligament interfaces in a subject by implanting an in vitro-prepared tissue between a detached tendon or ligament and a bone in a subject. The methods are particularly useful for regenerating bone-tendon interfaces of rotator cuff tendons, and thus, for restoring, partially or completely, rotator cuff joint function in a subject.

In some embodiments, the invention relates to an implantable article comprising an in vitro-prepared tissue comprising an outer border, an inner region and a slit, the slit extending along a length from the outer border to the inner region. The slit can be open at the outer border, and it can be substantially straight along the length from the outer border to the inner region. The slit can forms an opening having a substantially uniform width along the length from the outer border to the inner region.

The slit can have a width in range of 0.01 mm to 5.0 mm, more preferably in range of 0.1 mm to 2.5 mm, more preferably in range of 1.0 mm to 2.0 mm.

The in vitro-prepared tissue can contain a passage that adjoins the slit at the inner region, the width of the passage being in a range of greater than one times to five times the width of the slit. The in vitro-prepared tissue can form a circular passage that adjoins the slit at the inner region, and the inner region can be located approximately at the center of the in vitro-prepared tissue. The length from the outer border to the inner region can be in a range of 30% to 70% of the average length of the in vitro-prepared tissue. The length from the outer border to the inner region can be in a range of 45% to 55% of the average length of the in vitro-prepared tissue.

The in vitro-prepared tissue can be shaped as a disc, and the disc can have a diameter in a range of 5 mm to 10 mm. The in vitro-prepared tissue can be shaped in two dimensions as a closed geometric figure, and the closed geometric figure can have curved or straight sides. The in vitro-prepared tissue can have a substantially planar face.

The in vitro-prepared tissue can comprise fibroblasts, and the fibroblasts can be obtained from a donor and propagated in vitro. The fibroblasts can be dermal fibroblasts, ligament fibroblasts, tendon fibroblasts, or mucosal fibroblasts. The dermal fibroblasts can be foreskin-derived fibroblasts.

The in vitro-prepared tissue can further comprises a matrix protein selected from: type I collagen, type III collagen, fibronectin, and tenascin, and the matrix protein can be secreted by fibroblasts in the in vitro-prepared tissue. The in vitro-prepared tissue can further comprise a glycosaminoglycan selected from: versican, decorin, betaglycan, and syndecan, and the glycosaminoglycan can be secreted by fibroblasts in the in vitro-prepared tissue.

The in vitro-prepared tissue further can comprise a growth factor or cytokine selected from: PDGF-A, IGF, KGF, HBEGF, TGF-α, TGF-β1, TGF-β3, VEGF, FGF, G-CSF, IL-6, IL-8, Angiopoietin I, and HGF, and the growth factor or cytokine can be secreted by fibroblasts in the in vitro-prepared tissue. The in vitro-prepared tissue can comprise a diffusible form of VEGF or an extracellular matrix-binding form of VEGF. The in vitro-prepared tissue can comprise an extracellular matrix-binding form of FGF-2.

The in vitro-prepared tissue can comprise a scaffold composed of a biodegradable material, and the biodegradable material can be polyglycolic acid, polylactic acid, or a co-polymer thereof, or the biodegradable material can be polyglactin 910. The scaffold can be pre-coated with a naturally derived material or synthetic material. The naturally derived material can be collagen. The scaffold can be a mesh.

The in vitro-prepared tissue can have a thickness in a range of about 0.01 mm to about 2.0 mm, and it can be an in vitro-prepared dermal tissue. The in vitro-prepared tissue can have a longest dimension in a range of 5 mm to 50 mm, more preferably a longest dimension in a range of 5 mm to 25 mm.

In some embodiments, the invention relates to a cutting article comprising a solid support having a cutting edge, the cutting edge delineating an outer border and a slit, the slit being delineated by a first portion of the cutting edge extending inwardly along a length from the outer border and a second portion of the cutting edge extending inwardly along a length from the outer border, the first portion and second portion of the cutting edge being joined at an inner region. The cutting edge can be continuous, and the first portion of the cutting edge can be substantially parallel with the second portion of the cutting edge. The first portion of the cutting edge and the second portion of the cutting edge can each extend inwardly along a substantially straight path from the outer border to the inner region.

Other than at the inner region, the minimum distance between the first portion of the cutting edge and the second portion of the cutting edge can be in a range of 0.1 mm to 5.0 mm. The inner region can have a curvilinear shape, and the inner region can have a circular shape. The diameter of the circular shape of the inner region can be in a range of 0.1 mm to 5 mm.

The outer border can have a curvilinear shape, and the outer border can have a circular, oval, elliptical or polygonal shape. The diameter of the circular shape of the outer border can be in a range of 5 mm to 30 mm. The polygonal shape can be a hexagon, square or triangle.

In some embodiments, the invention relates to a cutting article comprising a solid support having a plurality of non-overlapping cutting edges, each cutting edge delineates an outer border and a slit, the slit being delineated by a first portion of the cutting edge extending inwardly along a length from the outer border and a second portion of the cutting edge extending inwardly along a length from the outer border, the first and second portions of the cutting edge being joined at an inner region.

In some embodiments, the invention relates to a method for preparing an implantable article comprising obtaining an in vitro-prepared tissue, and cutting the in vitro-prepared tissue to form an outer border and a slit, the slit extending along a length from the outer border to an inner region of the in vitro-prepared tissue.

In some embodiments, the invention relates to a method for preparing an implantable article comprising obtaining a scaffold, cutting the scaffold to form an outer border and a slit, the slit extending along a length from the outer border to an inner region of the in vitro-prepared tissue, and culturing mammalian cells on the scaffold under conditions that permit attachment of the mammalian cells to the scaffold and synthesis of extracellular matrix by the mammalian cells. The cutting can comprise depressing the cutting edge of a cutting article onto the scaffold or in vitro-prepared tissue.

In some embodiments, the invention relates to an implantable article prepared by the above methods.

In some embodiments, the invention relates to an implantable article comprising an in vitro-prepared tissue comprising a first outer border, a first inner region and a first slit, the first slit extending along a length from the first outer border to the first inner region; and a first support structure having a second outer border, a second inner region and a second slit, the second slit extending along a length from the second outer border to the second inner region; wherein the in vitro-prepared tissue is positioned adjacent to the first support structure such that the first slit is aligned with the second slit. The first slit can be open at the first outer border and the second slit is open at the second outer border. The first slit can be substantially straight along the length from the first outer border to the first inner region, and the second slit can be substantially straight along the length from the second outer border to the second inner region. The first slit can form an opening having a substantially uniform width along the length from the first outer border to the first inner region, and the second slit can forms an opening having a substantially uniform width along the length from the second outer border to the second inner region. The width of the first slit and the width of the second slit can be in range of 0.1 mm to 5 mm. The in vitro-prepared tissue can form a first passage that adjoins the first slit at the first inner region, and the width of the first passage can be in a range of equal to or greater than one times to five times the width of the first slit. The first support structure can form a second passage that adjoins the second slit at the second inner region, and the width of the second passage can be in a range of equal to or greater than one times to five times the width of the second slit.

The first passage can be aligned with the second passage, and the first passage and the second passage can have a curvilinear cross-sectional shape. The first inner region can be located approximately at the center of the in vitro-prepared tissue, and the second inner region can be located approximately at the center of the first support structure.

The length of the first slit can be 30% to 70% of the average length of the in vitro-prepared tissue, and the length of the second slit can be 30% to 70% of the average length of the first support structure

The in vitro-prepared tissue and/or the first support structure can be shaped as a disc. The disc can have a diameter in a range of 5 mm to 30 mm. The in vitro-prepared tissue and/or the first support structure can be shaped in two dimensions as a closed geometric figure, and the closed geometric figure can have curved or straight sides. The in vitro-prepared tissue can have a substantially planar face.

The first support structure can comprise an interior mesh, and the first support structure can comprise a first interior border that defines a first interior opening. The first interior border can contour the second outer border and the second slit.

The implantable article can further comprise a second support structure comprising a third outer border, a third inner region and a third slit, the third slit extending along a length from the third outer border to the third inner region, wherein the first support structure is attached to the second support structure such that the in vitro-prepared tissue is immobilized between the first support structure and the second support structure and the first slit, second slit and third slit are all aligned. The first slit can be open at the first outer border, the second slit can be open at the second outer border, and the third slit can be open at the third outer border. The second support structure can form a third passage that adjoins the third slit at the third inner region, and the width of the third passage can be in a range of equal to or greater than one times to five times the width of the third slit. The third passage can be aligned with the first passage and the second passage. The third passage can have a curvilinear cross-sectional shape.

The first support structure can have substantially the same shape as the second support structure. The first support structure can be joined with the second support structure such that the in vitro-prepared tissue is sandwiched between the first support structure and the second support structure. The first support structure can comprise at least one first connector and the second support structure can comprise at least one second connector, wherein the at least one first connector is shaped to mate with the at least one second connector. The at least one first connector can be mated with the at least one second connector. The at least one first connector can be positioned at the second outer border and the at least one second connector can be positioned at the third outer border.

The implantable article can further comprise at least one joining structure. The at least one joining structure can be a clamp that clamps the first support structure with the second support structure. The at least one joining structure can clamp the first support structure with the second support structure such that the in vitro-prepared tissue is sandwiched between the first support structure and the second support structure.

The first support structure can be composed of the same material as the second support structure. The second support structure can comprise a second interior mesh. The second support structure can comprise a second interior border that defines a second interior opening. The second interior border can contour the third outer border and the third slit.

In some embodiments the invention relates to a device for introducing an implantable article to a subject, the device comprising an elongated sheath having an distal opening, a distal internal cavity and a proximal internal cavity, the distal internal cavity being shaped to receive an implantable article through the distal opening, a plunger having a distal end and a proximal end, the distal end being adapted to interface with the implantable article in the distal internal cavity, the proximal end being movably fitted within the proximal internal cavity, and an actuator for moving the plunger axially within the sheath between a retracted position and an extended position, the actuator being coupled with the plunger at the proximal end, wherein movement of the plunger to the retracted position permits the implantable article to be received in the distal internal cavity, and movement of the plunger to the extended position causes the implantable article to be ejected from the distal internal cavity. The implantable article can be an implantable article described above.

The sheath can form a distal slit extending axially from the distal opening through at least a portion of the distal internal cavity. The distal slit can be configured to align with a slit of the implantable article. A portion of the actuator can be external to the sheath and pass through a proximal opening into the proximal internal cavity.

In some embodiments the invention comprises a device for introducing an implantable article to a subject, the device comprising an elongated sheath means having a distal internal cavity and a proximal internal cavity, the distal internal cavity being shaped to receive an implantable article through the distal opening, a plunger means having a distal end and a proximal end, the distal end being adapted to interface with the implantable article, the proximal end being movably fitted within the proximal internal cavity, and an actuator means for moving the plunger axially within the sheath between a retracted position and an extended position, the actuator being coupled at the proximal end of the plunger, wherein movement of the plunger to the retracted position permits the implantable article to be received in the distal internal cavity, and movement of the plunger to the extended position causes the implantable article to be ejected from the distal internal cavity. In some embodiments the invention comprises a system for minimally invasive surgery, the system comprising a device described above, and a cannula for accessing an implant site, the cannula having an elongated body defining a passage for receiving the device. The system can further comprise an implantable article described above.

In some embodiments the invention relates to a method for implanting an in vitro-prepared tissue between a detached tendon and a bone in a subject, the method comprising attaching a first portion of at least one suture to a detached tendon and attaching a second portion of the at least one suture to a bone in a subject; obtaining an implantable article comprising an in vitro-prepared tissue comprising an outer border, an inner region and a slit, the slit extending along a length from the outer border to the inner region; and positioning the implantable article such that the at least one suture fits transversely within the slit. The method can further comprise tensioning at least one suture so that the implantable article is mechanically compressed between the detached tendon and the bone at the tendon insertion site. The implantable article can be an implantable article described above.

The attaching and positioning can be performed during an open surgery on the subject, the attaching and positioning can be performed using a minimally-open surgical technique on the subject, or the attaching and positioning can be performed during a minimally invasive (arthroscopic) surgery on the subject.

In some embodiments the invention relates to a method for implanting an in vitro-prepared tissue between a detached tendon and a bone in a subject, the method comprising attaching a first portion of at least one suture to a detached tendon and attaching a second portion of the at least one suture to a bone in a subject; obtaining an implantable article comprising (a) an in vitro-prepared tissue comprising a first outer border, a first inner region and a first slit, the first slit extending along a length from the first outer border to the first inner region, and (b) a first support structure having a second outer border, a second inner region and a second slit, the second slit extending along a length from the second outer border to the second inner region, the in vitro-prepared tissue and first support structure being arranged such that the first slit is aligned with the second slit; and positioning the implantable article such that the at least one suture fits transversely within the first slit and second slit. The implantable article can an implantable article as described above.

The method for implanting an in vitro-prepared tissue between a detached tendon and a bone in a subject can further comprise percutaneously inserting a cannula for accessing the site between the detached tendon and the bone in a subject. The implantable article can be disposed within the distal internal cavity of the device, and the positioning can comprise inserting the device into the cannula, and causing the plunger to move to the extended position thereby ejecting the implantable article between the detached tendon and the bone such that the at least one suture fits transversely within the first slit and second slit.

In some embodiments the invention relates to a method for implanting an in vitro-prepared tissue between a detached tendon and a bone in a subject, the method comprising attaching a first portion of at least one suture to a detached tendon and attaching a second portion of the at least one suture to a bone in a subject; obtaining an implantable article comprising (a) an in vitro-prepared tissue comprising a first outer border, a first inner region and a first slit, the first slit extending along a length from the first outer border to the first inner region, (b) a first support structure having a second outer border, a second inner region and a second slit, the second slit extending along a length from the second outer border to the second inner region, and (c) a second support structure comprising a third outer border, a third inner region and a third slit, the third slit extending along a length from the third outer border to the third inner region, wherein the first support structure is attached to the second support structure such that the in vitro-prepared tissue is immobilized between the first support structure and the second support structure and the first slit, second slit and third slit are all aligned; and positioning the implantable article such that the at least one suture fits transversely within the first slit, second slit and third slit. The implantable article can be an implantable article as described above.

The method for implanting an in vitro-prepared tissue between a detached tendon and a bone in a subject can further comprise percutaneously inserting a cannula for accessing a site between the detached tendon and the bone in a subject. The method can further comprise obtaining a device as described above, wherein the implantable article is disposed within the distal internal cavity of the device, and wherein positioning can comprise inserting the device into the cannula, and causing the plunger of the device to move to the extended position thereby ejecting the implantable article between the detached tendon and the bone such that the at least one suture fits transversely within the first slit, second slit and third slit.

The method for implanting an in vitro-prepared tissue between a detached tendon and a bone in a subject can further comprise tensioning at least one suture so that the implantable article is mechanically compressed between the detached tendon and the bone at the tendon insertion site.

In some embodiments, the invention further comprises a method for implanting an in vitro-prepared tissue between a detached ligament and a bone in a subject, the method comprising attaching a first portion of at least one suture to a detached ligament and attaching a second portion of the at least one suture to a bone, obtaining an implantable article comprising an in vitro-prepared tissue comprising an outer border, an inner region and a slit, the slit extending along a length from the outer border to the inner region, and positioning the implantable article such that the at least one suture fits transversely within the slit. The implantable article can be an implantable article as described above. The implantable article can be an implantable article as described above.

In some embodiments, the invention relates to an implantable article comprising an in vitro-prepared tissue comprising an outer border, an inner region and a slit, the slit extending along a length from the outer border to the inner region, wherein the in vitro-prepared tissue comprises fibroblasts.

In some embodiments, the invention relates to a method for implanting an in vitro-prepared tissue between a detached tendon and a bone in a subject, the method comprising attaching a first portion of at least one suture to a detached tendon and attaching a second portion of the at least one suture to a bone in a subject; obtaining an implantable article comprising (a) an in vitro-prepared tissue comprising a first outer border, a first inner region and a first slit, the first slit extending along a length from the first outer border to the first inner region, and (b) a first support structure having a second outer border, a second inner region and a second slit, the second slit extending along a length from the second outer border to the second inner region, the in vitro-prepared tissue and first support structure being arranged such that the first slit is aligned with the second slit; and positioning the implantable article such that the at least one suture fits transversely within the first slit and second slit, wherein the in vitro-prepared tissue comprises fibroblasts.

In some embodiments, the in vitro-prepared dermal tissue comprises dermal fibroblasts on a scaffold, and the scaffold is absorbed by the subject while the subject does not reject the in vitro-prepared dermal tissue. In some embodiments, the dermal fibroblasts remain at the internal, bone-tendon interface after the scaffold is absorbed. Thus, in some aspects, methods are provided for regenerating a bone-tendon interface in a subject by implanting an in vitro-prepared dermal tissue between a detached tendon and a bone in a subject. The methods are particularly useful for regenerating bone-tendon interfaces of rotator cuff tendons, and thus, for restoring, partially or completely, rotator cuff joint function in a subject.

According to some aspects, methods are provided for maintaining exogenous, viable fibroblasts between a detached tendon and a bone in a subject. In some embodiments, the methods comprise: implanting an in vitro-prepared dermal tissue that comprises viable fibroblasts between a detached tendon and a bone in a subject. In some embodiments, the in vitro-prepared dermal tissue comprises allogeneic fibroblasts. In some embodiments, the in vitro-prepared dermal tissue comprises autologous fibroblasts. In some embodiments, the methods further comprise: isolating the autologous fibroblasts from the subject; and preparing the in vitro-prepared dermal tissue using the autologous fibroblasts.

According to some aspects, methods are provided for delivering exogenous cytokines and/or growth factors to a damaged bone-tendon interface. In some embodiments, the methods comprise: implanting an in vitro-prepared dermal tissue at the damaged bone-tendon interface in the subject. In some embodiments, the in vitro-prepared dermal tissue comprises an exogenous growth factor or cytokine selected from: PDGF-A, IGF, KGF, HBEGF, TGF-α, TGF-β1, TGF-β3, VEGF, G-CSF, IL-6, IL-8, Angiopoietin I, HGF and SPARC. In some embodiments, the in vitro-prepared dermal tissue comprises diffusible and extracellular matrix-binding forms of VEGF. In some embodiments, the exogenous growth factor or cytokine is secreted by fibroblasts present in the in vitro-prepared dermal tissue.

According to some aspects of the disclosure, methods of regenerating a bone-tendon interface in a subject are provided. In some embodiments, the methods comprise implanting an in vitro-prepared tissue between a detached tendon and a bone in a subject. According to other aspects of the disclosure, methods of evaluating regeneration of a bone-tendon interface are provided. In some embodiments the methods comprise detaching a tendon at a bone-tendon interface of a subject and implanting an in vitro-prepared tissue between the detached tendon and the bone of the bone-tendon interface in the subject.

According to some aspects, methods are provided for promoting wound healing at a damaged bone-tendon interface in a subject. In some embodiments, the methods comprise: implanting an in vitro-prepared dermal tissue at the damaged bone-tendon interface in the subject.

In certain embodiments of the methods, the in vitro-prepared tissue is an in vitro-prepared dermal tissue. In certain embodiments of the methods, the in vitro-prepared dermal tissue is DERMAGRAFT (Interactive Wound Dressing). In some embodiments of the methods, the in vitro-prepared tissue comprises fibroblasts. In certain embodiments, the fibroblasts are dermal fibroblasts. In one embodiment, the dermal fibroblasts are human foreskin fibroblasts. In certain embodiments, the in vitro-prepared tissue comprises stromal cells. In certain embodiments, the in vitro-prepared tissue does not comprise hair follicles, macrophages, or lymphocytes.

In some embodiments of the methods, the in vitro-prepared tissue further comprises a matrix protein selected from: Type I Collagen, Type III Collagen, Fibronectin, and Tenascin. In certain embodiments, the matrix protein is secreted by cells in the in vitro-prepared tissue. In other embodiments, the matrix is exogenously added to the in vitro-prepared tissue.

In some embodiments of the methods, the in vitro-prepared tissue further comprises a glycosaminoglycan selected from: versican, decorin, betaglycan, and syndecan. In certain embodiments, the glycosaminoglycan is secreted by cells in the in vitro-prepared tissue. In other embodiments, the glycosaminoglycan is exogenously added to the in vitro-prepared tissue.

In some embodiments of the methods, the in vitro-prepared tissue further comprises a growth factor selected from: PDGF-A, IGF, KGF, HBEGF, TGF-α, TGF-β1, TGF-β3, VEGF, G-CSF, Angiopoietin I, HGF and SPARC. In certain embodiments, the growth factor is secreted by cells in the in vitro-prepared tissue. In other embodiments, the growth factor is exogenously added to the in vitro-prepared tissue. In some embodiments, the in vitro-prepared tissue comprises diffusible and extracellular matrix-binding forms of VEGF.

In some embodiments of the methods, the in vitro-prepared tissue comprises a scaffold formed into a three-dimensional structure. In certain embodiments, the in vitro-prepared tissue substantially envelops the scaffold. In certain embodiments, the three-dimensional structure comprises interstitial spaces bridged by fibroblasts or stromal cells. In one embodiment, fibroblasts or stromal cells synthesize extracellular matrix that resides in the interstitial spaces. In some embodiments, the scaffold is composed of a biodegradable material. In certain embodiments, the biodegradable material is polyglycolic acid, polylactic acid, or a co-polymer thereof. In one embodiment, the biodegradable material is polyglactin. In some embodiments, the scaffold is pre-coated with collagen. In one embodiment, the scaffold is a mesh. In some embodiments, a scaffold may be referred to equivalently as a substrate.

In some embodiments of the methods, implanting comprises positioning the in vitro-prepared tissue on a tendon insertion site of the bone and positioning the detached tendon on the in vitro-prepared tissue. In certain embodiments, positioning the in vitro-prepared tissue comprises substantially covering the tendon insertion site with the in vitro-prepared tissue. In certain embodiments, the in vitro-prepared tissue is a sheet. In one embodiment, the sheet has length of about 7.5 cm and a width of about 5.0 cm. In one embodiment, the sheet has a thickness in a range of about 0.01 mm to about 2.0 mm. In some embodiments, the methods further comprise cutting the sheet to a size slightly larger than the tendon insertion site prior to positioning the in vitro-prepared tissue on the tendon insertion site. In one embodiment, the in vitro-prepared tissue is cut to a length of about 2.0 cm and a width of about 1.5 cm. In some embodiments, the methods further comprise folding the sheet to form at least two layers and positioning the folded sheet on the tendon insertion site. In one embodiment, the sheet is folded to form three layers and the three layers are positioned such that one outer layer contacts the bone and the other outer layer contacts the detached tendon.

In some embodiments of the methods, implanting further comprises fastening the detached tendon to the bone so that the in vitro-prepared tissue is mechanically compressed between the detached tendon and the bone at the tendon insertion site. In certain embodiments, fastening comprises attaching a first portion of at least one suture to the detached tendon and attaching a second portion of the at least one suture to the bone. In certain embodiments, the second end of the at least one suture is connected with a fastener and wherein attaching the second end of the at least one suture to the bone comprises connecting the fastener to the bone. In one embodiment, connecting the fastener to the bone comprises drilling a hole in the bone at a location proximal to the tendon insertion site and anchoring the fastener in the hole. In another embodiment, fastening further comprises tensioning the at least one suture so that at least a portion of the in vitro-prepared tissue is mechanically compressed between the detached tendon and the bone at the tendon insertion site of the bone.

In some embodiments of the methods, the in vitro-prepared tissue is cryopreserved and the methods further comprise thawing the cryopreserved in vitro-prepared tissue prior to implanting. In certain embodiments, thawing is performed at an average temperature of 34° C. to 37° C. In certain embodiments, thawing is completed within about 2 minutes following removal of the in vitro-prepared tissue from cryopreservation.

In some embodiments of the methods, the tendon is a tendon of a rotator cuff muscle. In certain embodiments, the tendon is selected from subscapularis tendon, supraspinatus tendon, infraspinatus tendon, and teres minor tendon. In one embodiment, the bone is a humerus. In another embodiment, the tendon insertion site is on the humeral head of the humerus. In another embodiment, the tendon insertion site is on the lesser tuberosity on the anterior aspect of the humeral head. In yet another embodiment, the bone tendon interface is at the greater tuberosity of the humeral head.

In some embodiments, the methods further comprise performing in vivo imaging of the bone-tendon interface to determine the quality or extent of regeneration of the bone-tendon interface. In some embodiments, the methods further comprise obtaining a tissue sample comprising at least a portion of the bone and tendon of the bone-tendon interface at a predetermined time after implanting the in vitro-prepared tissue. In certain embodiments, the methods further comprise performing a histological assay on the tissue sample to determine the extent of regeneration of the bone-tendon interface. In certain embodiments, the extent of regeneration of the bone-tendon interface is determined, at least in part, by comparing the results of the histological assay with an appropriate reference. In other embodiments, the methods further comprise performing a biomechanical test on the tissue sample to determine a mechanical property of the bone-tendon interface. In certain embodiments, the in vitro-prepared tissue comprises cells derived from a species other than the species of the subject. In one embodiment, the cells are human cells. In certain embodiments, the in vitro-prepared tissue comprises cells comprising an exogenous nucleic acid. In some embodiments, the methods further comprise identifying the cells in the tissue sample. In some embodiments, identifying the cells comprises detecting a marker indicative of the species from which the cells were derived. In other embodiments, identifying the cells comprises detecting a marker indicative of the presence of the exogenous nucleic acid.

### BRIEF DESCRIPTION OF DRAWINGS

FIG. 1 depicts an exemplary in vitro-prepared tissue. FIG. 1B depicts the view from the orientation identified in FIG. 1A by two parallel arrows labeled “1B”.

FIG. 2 depicts an exemplary support structure. FIG. 2B depicts the view from the orientation identified in FIG. 2A by two parallel arrows labeled “2B”. FIG. 2C depicts the view from the orientation identified in FIG. 2A by two parallel arrows labeled “2C”. FIG. 2D depicts the view from the orientation identified in FIG. 2A by two parallel arrows labeled “2D”.

FIG. 3A-C depict an exemplary implantable article comprising two support structures having interior openings.

FIG. 4A-C depict an exemplary implantable article comprising two support structures having interior mesh portions.

FIG. 5A-C depict an exemplary implantable article having a center passage for a suture.

FIG. 6A-B depict an exemplary implantable article comprising two support structures having integral joining structures.

FIG. 7 depicts an exemplary device for producing implantable articles.

FIGS. 8A-B depict an exemplary system for minimally invasive surgery comprising a device for introducing an implantable article to a subject and a cannula for accessing an implant site.

FIGS. 9A-D depict exemplary methods for repairing a bone-tendon interface using a system for minimally invasive surgery comprising a device for introducing an implantable article to a subject and a cannula for accessing an implant site.

FIG. 10 shows the generation of DERMAGRAFT, a human Type I collagen-rich tissue matrix.

FIG. 11A shows in a sheep model the generation of bone tunnels on the head of the humerus following detachment of the infraspinatus tendon. FIG. 11B shows the addition of DERMAGRAFT onto the decorticated head of the humerus. FIG. 11C shows placement of sutures through the bone tunnels to reattach the tendon and hold the DERMAGRAFT in place. FIG. 11D shows a reattached tendon with the DERMAGRAFT interposed between the tendon and the bone.

FIG. 12 shows a harvested shoulder from a sheep model of rotator cuff repair that is split down the middle, revealing the level of retraction of the tendon end (white arrows) from the original attachment site (black arrows).

FIG. 13A shows reparative neo-tendon with increased neo-vessels running parallel to the tissue fibers (arrows) 4X HE (Sheep #1). FIG. 13B shows the junction of retracted mature tendon (left) and the neo-tendon (right). (20X HE.) The insert of FIG. 13B shows neo-tendon with regular periodicity. 4X PR (Sheep #1). FIG. 13C shows the junction of retracted mature tendon (left) and neo-tendon (right). (10X TB.) (Sheep #1). FIG. 13D shows the presence of Sharpey's fibers (arrow) anchoring the neo-tendon to the bone. 10X, HE. (Sheep #9). FIG. 13E shows a higher magnification of the insert in FIG. 4D showing Sharpy's fibers. 20X, HE. FIG. 13F shows the areas with clefts/voids (white arrows) showing osteoclastic resorption (black arrows). 10X. HE (Sheep #1). FIG. 13G shows that at the bone-tendon interface there were areas of well defined Sharpeys fibers (arrows) anchoring the neo-tendon to the bone with active bone formation, including the presence of reversion lines and lining osteoblasts.

FIG. 14A shows a human shoulder joint with a torn supraspinatus muscle. FIG. 14B is an intraoperative picture showing the infraspinatus tendon of the sheep shoulder.

FIG. 15 shows an intraoperative picture of the sheep shoulder joint model. The infraspinatus tendon has been detached from the humerus then reattached, with a matrix placed under the Mason-Allen suture pattern used to reattach the tendon.

FIG. 16 depicts a bisection line along the supraspinatus muscle. Two histological sections are obtained each from the cranial and the caudal aspect.

FIG. 17A shows specimen positioning in the MiniBionix MTS prior to testing. FIG. 17B shows an expanded view of the sample corresponding to the inset in FIG. 17A.

### DETAILED DESCRIPTION OF INVENTION

In vitro prepared tissues are provided herein for implantation between a detached tendon or ligament and bone. The in vitro prepared tissues are useful for delivering cells, growth factors and/or extracellular matrix molecules to stimulate tissue generation. Thus, an in vitro-prepared tissue can be implanted between a detached tendon or detached ligament and a bone in a subject to stimulate reattachment of the tendon or ligament to the bone. According to some aspects of the invention, implantable articles are provided that comprise in vitro prepared tissue for implantation between a detached tendon or ligament and bone. Accordingly, in some aspects, the implantable articles are useful for delivering cells, growth factors and/or extracellular matrix molecules to stimulate tissue generation. Implantable articles are designed to interface with a suture connecting a detached tendon or ligament with bone. Interfacing with the suture facilitates immobilization of the article at the implant site. Immobilization of the article is often achieved by compressing the article between a detached tendon or ligament and bone by tensioning of one or more sutures.

The implantable articles are suitable for delivery using any one of a variety of surgical techniques, including, for example, minimally invasive surgery, semi-open surgery and open surgery. The size of the implantable articles are larger or smaller than the percutaneous access portal for arthrotomy or arthroscopic procedures of the shoulder. In one embodiment, the implantable article is transferred to the implant at a torn rotator cuff tendon site via an access port ranging from approximately 8 mm to 12 mm diameter wide, created and maintained open with a canula used during arthroscopic approaches. In another embodiment, an implantable article is transferred to its implant site at a torn rotator cuff tendon through an incision that can range from 1-12 cm or greater that serves as approximately 12 mm incision that serves as an access portal for an arthrotomy approach to open, mini-open, or arthroscopy. The sizes provided are not meant to limit the potential sizes of the implant that can be delivered. In the case of an open surgical procedure, the implant could be between 0.5 and 4 cm or greater for delivery to the site of treatment. Accordingly, methods are provided herein for implanting in vitro-prepared tissues to stimulate the attachment of tendon or ligament to bone. Methods of the invention provide improved recovery time and decreased risk of re-injury compared with existing methods. Typically, a repair is deemed successful if the subject regains all or a portion of the function of the tendon or ligament. However, in some cases, e.g., where the subject has a sedentary lifestyle or where the damage is particularly severe, any degree of restoration of tendon or ligament stability may be adequate.

Implantable articles of the invention typically comprise one or more in vitro-prepared tissues formed to interface with a suture at an implant site. The term “in vitro-prepared tissue,” as used herein, refers to a tissue that has been synthesized in a controlled environment outside of a living organism. Typically, an in vitro-prepared tissue is synthesized by cells. The cells may be allogeneic, xenogenic, syngenic or autologous with respect to the subject within whom the article is to be implanted. For example, cells may be obtained from a subject and used to synthesize an in vitro-prepared tissue that is implanted back into the subject. Cells may be obtained from a donor and used to synthesize an in vitro-prepared tissue that is implanted into another subject. Cells may be obtained from a donor of a particular species and used to synthesize an in vitro-prepared tissue that is implanted into a subject of another species.

An in vitro-prepared tissue may or may not comprise living cells, depending on intended use of the tissue. For an in vitro-prepared tissue comprising cells, the cells may be kept viable, for example, by maintaining the tissue under suitable tissue culture conditions and/or by cryopreserving the tissue under conditions that preserve cell membrane integrity and enable cell growth after thawing. Alternatively, cells of an in vitro-prepared tissue may be killed or eliminated using any appropriate method known in the art, e.g., by cell lysis.

Typically, the in vitro-prepared tissue comprises an underlying scaffold that provides a structure for cell attachment and that defines a basic shape of the in vitro-prepared tissue. As used herein, the term “scaffold” refers to a three-dimensional structure on which cells may attach, grow and synthesize tissue. A scaffold may be composed of natural components, synthetic components or a combination thereof. Typically, a scaffold is biocompatible and biodegradable. Methods for producing scaffolds are well known in the art and exemplary methods are disclosed herein. As will be appreciated by the skilled artisan, scaffolds may be designed and constructed to tune their mechanical properties and degradation rates (Encyclopedia of Biomaterials and Biomedical Engineering, Volume 3 By Gary E. Wnek, Gary L. Bowlin.) The scaffold should be porous enough to enable metabolism of the cells seeded and proliferating within the scaffold, and should have sufficient tensile and compressive strengths to support extracellular matrix deposition by the cells on the scaffold. The scaffold should further allow for physical manipulation of the in vitro-prepared tissue to support its delivery into the site of treatment.

Implantable articles of the invention often comprise one or more support structures. As used herein the term “support structure” refers to a component that augments an in vitro-prepared tissue to achieve a desired form, shape, and/or mechanical property, e.g., rigidity, flexibility, strength. A support structure may be composed of natural components, synthetic components or a combination thereof. Typically, a support structure is biocompatible and/or biodegradable. The support structure can be of a rigid design to give the implantable article a substantially fixed shape under implantation conditions or can be of a flexible design that facilities deformation of the implantable article during implantation.

The support structure may be any shape (e.g. disc or rectangular in shape). The support structure should be rigid enough to hold its shape during arthroscopic delivery through a port and to the implantable article's implantation site under standard irrigation forces, or if deformed during the delivery process, the support structure should have the capabilities to regain its approximate original shape as described below. In some embodiments, the support structure should be flexible enough to allow folding of it and the in vitro prepared tissue surrounded by the support structure such that the composite implantable article can transit through a port with smaller dimensions than the implantable article, in its unfolded state.

An in vitro-prepared tissue and/or its underlying scaffold may alone achieve a desired form, shape, and/or mechanical property of the implantable article, in which case, a support structure may not be required.

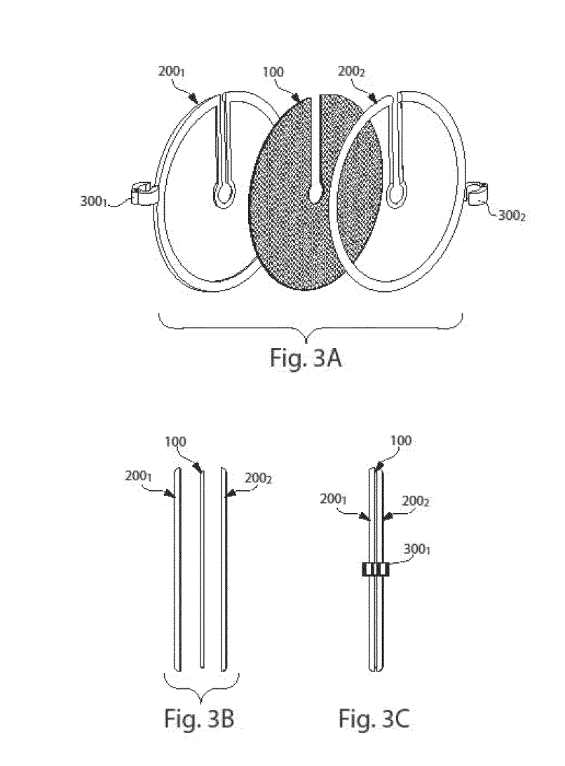

In some cases, often where either single or multiple support structures are used, the implantable articles comprise one or more “joining structures.” As used herein the term “joining structure” refers to a component that joins together two or more other components. For example, a joining structure may join together two or more support structures, may join together one or more support structures with one or more in vitro-prepared tissues, or may join together two or more in vitro-prepared tissues. Any of a variety of joining structures may be used, including, for example, Velcro, snap connectors, buttons, screws, clamps, sutures, and various other fasteners. For example, male and female snap connectors may be used to join together two support structures. Alternatively, two support structures that comprise suture holes around the support structures' circumference are used to join multiple support structures together via sutures, immobilizing the in vitro prepared tissue between the support structures. As another example, one or more compression clamps may be used to hold together multiple support structures. In some embodiments, one or more joining structures join together a pair of support structures such that an in vitro-prepared tissue is sandwiched between the support structures (See, e.g., FIG. 3). In some cases, where a single support structure is used, the implantable article comprises a single structure and an in vitro prepared tissue are held together by one or more joining structures, as an example, sutures, that either pass through in the support structure or wrap around the support structure.

These configurations immobilizes the in vitro-prepared tissue between the support structures.

In some embodiments, where single or multiple support structures are used, the support structures themselves comprise an integral joining structure (e.g., snaps, Velcro, connectors, etc.) in which case, they may be connected together without the need for separate joining structure(s).

### Implantable Articles

Implantable articles of the invention typically comprise an outer border, an inner region and a slit. The slit extends along a length from the outer border to the inner region. The slit is typically open at the outer border of the article so that a suture at a joint repair site can slide into the slit. Interfacing with a suture in this manner facilitates positioning and immobilization of the implantable article between a tendon or ligament and bone, particularly in the context of minimally invasive surgery. When the implantable article slides onto a suture between a detached tendon or ligament and bone, tensioning of the suture causes the tendon or ligament to compress the implantable article between the tendon or ligament and the bone. Compression of the implantable article between the tendon or ligament and the bone serves to keep the article in place. Because the implantable article comprises an in vitro-prepared tissue enriched in growth factors and matrix proteins, the presence of the article between the detached tendon or ligament and bone stimulates the repair process.

The slit of an implantable article can have a variety of shapes and dimensions provided that it has a width sufficient to allow a suture to slide into (or pass through) the slit. As used herein, the term “slit” refers to an elongated passage through a material, such as, e.g., an in vitro-prepared tissue, a support structure, etc. Typically the width of the slit is slightly larger than the width of a suture such that the suture can slide into the slit. The slit is also usually open at a region in the outer border of the material. Often, the slit is substantially straight along the length from the outer border to the inner region, but it need not be. For example, hook-shaped or curved slits are also possible. The slit may form an opening having a substantially uniform width along the length from the outer border to the inner region, or the width may be irregular along the length. The width of the slit is typically in a range of 0.1 mm to 1 mm. In some embodiments, the width of the slit may be in the range of 0.01 mm to 1 mm or more.

A passage for a suture may be provided at an inner region of the implantable article. A passage may be provided alone or in combination with a slit. Thus, in some embodiments, only a passage for a suture is provided at an inner region of the implantable article. Typically, however, a passage is provided that adjoins a slit at an inner region of an implantable article. The passage may be located approximately at the center of the article, or at another position of the article, e.g., near the periphery of the article. The length from the outer border to the passage may be in a range of 30% to 70% of the average length of the article. The length from the outer border to the passage may be in a range of 45% to 55% of the average length of the article.

An implantable article is typically positioned at an implant site such that a suture is located in a passage at the inner region. The longitudinal axis of the suture may be approximately perpendicular with the longitudinal plane of the article. The passage cross section may be any of a variety of shapes, including, for example, a circle. Typically the width of the passage is in a range of about 0.2 mm to about 2 mm in diameter. Where a passage and slit are provided, the width of the passage is often slightly larger than the width of the slit. Typically the width of the passage is in a range of greater than one times to five times the width of the slit.

It should be appreciated that an implantable article may be immobilized at an implantation site in a variety of ways. In particular, an implantable article may interface with one or more sutures at the implantation site. In some embodiments, one or more sutures extend through a single passage of an implantable article. In certain embodiments, multiple sutures extend through multiple passages of an implantable article. Accordingly, an implantable article may have one or more slits, one or more passages, or combinations of one or more slits and one or more passages. In some embodiments, a slit extends from an outer border to the center of the article. In certain embodiments, a slit extends from an outer border to a region near the periphery of the article. Likewise, a passage may be present at the center of the article or may be present at a region near the periphery of an article.

An implantable article may be any of a variety of shapes. For example, an implantable article may be shaped in two dimensions as a closed geometric figure, it being appreciated that the closed geometric figure is interrupted by the slit. When used for minimally invasive surgery, the implantable article is typically shaped and sized in a way that is conducive to use with minimally invasive surgical equipment (e.g., an arthroscope, or other device for delivering an implantable article). For open surgery applications, the dimensional and size requirements of an implantable article are often less restrictive compared with minimally invasive surgeries.

An implantable article may comprise an in vitro-prepared tissue shaped in two dimensions as a closed geometric figure, it again being appreciated that the closed geometric figure is interrupted by the slit. Examples of closed geometric figures for the implantable article and the in vitro-prepared tissue include curved (e.g., circle, oval, ellipse) and linear (e.g., polygon) (e.g., triangle, square, pentagon, etc.) figures. The figures can be symmetrical or asymmetrical, and they can have curved or straight sides. Often the implantable article comprises an in vitro-prepared tissue that has a substantially planar face. In some embodiments, where the implantable article comprises a slit, the article may be deformed such that material adjacent to the slit overlaps to create a shape (e.g., a conical shape that can be passed through an arthroscope).

Typically the implantable article has an aspect ratio of a thin object. The implantable article may have a longest dimension (e.g., length, width, diameter) in a range of 5 mm to 10 mm, 10 mm to 15 mm, 15 mm to 20 mm, 20 mm to 25 mm, 25 mm to 30 mm, 30 mm to 35 mm, 35 mm to 40 mm, 40 mm to 45 mm, 45 mm to 50 mm, or more. The implantable article may have a longest dimension of up to 5 mm, 10 mm, 15 mm, 20 mm, 25 mm, 50 mm or more. The ratio of the longest dimension to shortest dimension (the aspect ratio) of the implantable article may be about 2000 to 1, about 1000 to 1, about 500 to 1, about 200 to 1, about 100 to 1 or about 10 to 1. The shortest dimension (thickness) may be in a range of about 0.05 mm to about 0.1 mm, about 0.1 mm to about 0.5 mm, about 0.5 mm to about 1 mm or about 1 mm to about 5 mm. The shortest dimension (thickness) may be about 0.05 mm, about 0.1 mm, about 0.2 mm, about 0.5 mm, about 1 mm, about 5 mm or more.

It will be appreciated that the shape and form of an implantable article will often be determined by the shape and form of its component parts as well as the manner in which the component parts are assembled, joined, or connected together. For example, an implantable article may comprise (i) an in vitro-prepared tissue comprising an outer border, an inner region and a slit, and (ii) one or more support structures. The support structure(s) may have an outer border, an inner region and a slit. The slit of the support structure(s) and the slit of the in vitro-prepared tissue are typically aligned (See, e.g., FIGS. 3-5). Alignment of the slits ensure that a suture can slide into the slits for positioning of the implantable article at a repair site. Thus, the dimension and shape of the slit of a support structure is typically similar or identical to the dimension and shape of the slit of a corresponding in vitro-prepared tissue. While the slit in the in vitro-prepared tissue may be produced during the generation of the in vitro-prepared tissue, the slit may also be created after the in vitro-prepared tissue has been combined with the support structure, using a separate cutting device to make the slit in the in vitro-prepared tissue.

It will also be appreciated that the mechanical properties of an implantable article may be determined by the mechanical properties of its component parts as well as the manner in which the component parts are assembled, joined, or connected together. Furthermore, any of the implantable articles and any of the component parts of the implantable articles disclosed herein can be of a rigid or flexible design.

### Cutting Articles

The invention, in some aspects, provides cutting articles, which are useful for cutting in vitro-prepared tissues and other components of the invention. A typical cutting article comprises a solid support having a cutting edge that delineates an outer border and may also include a slit, although a separate cutting article may be used to generate the outer border and the slit. The slit is delineated by a first portion of the cutting edge that extends inwardly along a length from the outer border and a second portion of the cutting edge that extends inwardly along a length from the outer border. The first portion and second portion of the cutting edge joined at the inner region. To delineate a slit having a uniform width, the first portion of the cutting edge is substantially parallel with the second portion of the cutting edge. Other than at the inner region where the first and second portions meet, the minimum distance between the first portion of the cutting edge and the second portion of the cutting edge may be in a range of 0.1 mm to 0.5 mm, 0.5 mm to 1 mm, 1 mm to 2 mm, or more. To define a straight slit, the first portion of the cutting edge and the second portion of the cutting edge each extend inwardly along a substantially straight path from the from the outer border to the inner region.

In some embodiments, a cutting article comprises a solid support having a cutting edge that delineates an outer border, a passage at an inner region, and a slit. In some embodiments, a cutting article comprises a solid support having one or more cutting edges that delineate an outer border and a passage at an inner region.

The cutting edge may delineate a passage having a curvilinear shape (e.g., a circular shape), at the inner region. The diameter of the circular shape of the passage may be in a range of up to 0.5 mm to 1 mm, 1 mm to 2 mm, 2 mm to 4 mm, or more. The cutting edge may define an outer border having any shape suitable for use in the methods of the invention. The cutting edge may define as an example an outer border having a curvilinear shape (e.g., circular, oval, elliptical) or polygonal shape (e.g., hexagonal, rectangular, square, triangular). The longest dimension (e.g., length, width, diameter) of an area circumscribed by the outer border may be in a range of 5 mm to 10 mm, 10 mm to 15 mm, 15 mm to 20 mm, 20 mm to 25 mm, 25 mm to 30 mm, 30 mm to 35 mm, 35 mm to 40 mm, 40 mm to 45 mm, 45 mm to 50 mm, or more. The longest dimension (e.g., length, width, diameter) of an area circumscribed by the outer border may be up to 5 mm, 10 mm, 15 mm, 20 mm, 25 mm, 30 mm, 35 mm, 40 mm, 45 mm, 50 mm, or more. For cutting a plurality of in vitro-prepared tissues together, an article for cutting an in vitro-prepared tissue may comprise a solid support having a plurality of non-overlapping cutting edges. Each cutting edge of the plurality may delineate an outer border and a slit. Each slit may be defined by a first portion of each cutting edge extending inwardly along a length from the outer border and a second portion of each cutting edge extending inwardly along a length from the outer border. The first and second portions of each cutting edge may be joined at the inner region. The outer border of each cutting edge in the plurality may or may not have identical shapes. While a plurality of cutting edges may be created, single cutting edges may also be used to cut single pieces of support structure out of a sheet of support material. Single or a plurality of cutting edges may also be used to cut one or more implantable articles, respectively, containing a) a plurality of layered support structures within which in vitro prepared tissue is layered or b) a single support structure layered with an in vitro prepared tissue.

In vitro-prepared tissues are often prepared initially as sheets. Cells are seeded on a scaffold, placed under conditions that facilitate cell growth and attachment to the scaffold and extracellular matrix synthesis, thereby producing an in vitro-prepared tissue. Before or after seeding of the cells, the scaffold may be cut using a cutting article of the invention to a form and size conducive to use in the methods disclosed herein (e.g., to a form having an outer border and slit). In some cases, an established in vitro-prepared tissue is cut to a form and size conducive to use in the methods disclosed herein. The cutting instruments used to cut the in vitro-prepared tissue may constitute a single cutting instrument that provides for the cutting of the outer form and the slit, or separate cutting instruments that are used to cut the outer form and to cut the slit.

### In Vitro-Prepared Tissues

An in vitro-prepared tissue may be synthesized by any of a variety of cells, including cells derived from skin tissue, such as fibroblasts, or cells derived from other tissues (e.g., tendon, ligament, synovium, muscle, mucosa, bone marrow) that are capable of producing natural products that are found in wound sites and that contribute to wound healing and/or tissue repair. In some cases these cells may be fibroblasts or other cell types such as endothelial cells or stem cells that are capable of either directly producing factors involved in wound healing, or are capable of differentiating into a cell type, or being induced to differentiate into a cell, that is capable of producing products that are involved in wound healing. Cell lines and genetically-modified cells may also be used provided that they are capable of producing components involved in wound healing and or tissue regeneration. Examples of cells that may be utilized to produce an in vitro-prepared tissue, include, but are not limited to, fibroblasts, stromal cells (e.g., marrow-derived stromal cells), and mesenchymal stem cells. When stromal cells or mesenchymal stem cells are used, it may be necessary, or desirable, to first induce the cells to differentiate or to separate out a subpopulation of cells that exhibit a fibroblastic phenotype. Subpopulations of cells may be separated by any of a variety of methods known in the art, including, for example, by FACS, by eliminating cells that do not attach to a substrate over a predetermined period of time, or by selecting cells that bind to a certain epitope which is characteristic of skin tissue (e.g., an RGD-peptide). In some embodiments, the cells of the in vitro-prepared tissue comprise fibroblasts. In some embodiments, the cells of the in vitro-prepared tissue consist only of fibroblasts. In some embodiments, the cells of the in vitro-prepared tissue do not comprise keratinocytes, endothelial cells, or Langerhans cells. In some embodiments, the in vitro-prepared tissue comprises synovium, cartilage, bone, tendon, ligament, or muscle producing cells.

In some embodiments, the in vitro-prepared tissue does not comprise living cells. For example, the in vitro-prepared tissue may be subjected to storage conditions that are not suitable for the maintenance of viable cells, as is done for the product TransCyte, which is a Human Fibroblast Derived Temporary Skin Substitute.

In vitro-prepared tissue typically comprises a variety of different extracellular matrix proteins, including those that are commonly found in healing wounds and/or skin. Non-limiting examples of such proteins include collagen, including any one of collagen Type I to XIII, elastin, laminin, fibronectin, and tenascin. In vitro-prepared tissue also typically comprises one or more glycosaminoglycans that are commonly found in normal tissues and in healing wounds or in sites of active tissue repair and remodeling. Non-limiting examples of such glycosaminoglycans are selected from: chondroitin sulfate, dermatan sulfate, keratan sulfate, heparin, heparan sulfate, hyaluronan, versican, decorin, betaglycan, and syndecan. Certain growth factors and cytokines are also often present in in vitro-prepared tissue. Examples of such growth factors and cytokines include, for example, IL-8, IL-6, IL-1α, PDGF-A, IGF, KGF, HBEGF, TGF-α, TGF-β1, TGF-β3, VEGF, G-CSF, Angiopoietin I, acidic FGF, basic FGF and HGF. In particular, in vitro-prepared tissue often includes components (e.g., growth factors) that stimulate angiogenesis. For example, in vitro-prepared tissue typically comprises diffusible and/or extracellular matrix-binding forms of VEGF, or basic FGF. While it is to be appreciated that matrix proteins, glycosaminoglycans, growth factors, and other compounds present in the tissue are typically expressed from the cells that are used to produce the tissue in vitro, exogenous sources may also be used. For example, soluble growth factors, such as, for example, TGF-β1, TGF-β3, VEGF, PDGF (PDGF-AA, -BB, -AB, -CC, -DD) may be exogenously added to the tissue (e.g., as recombinant proteins or proteins isolated from natural sources) and remain resident in the tissue. Extracellular matrix proteins may also be supplied exogenously. For example, collagen may be supplied as a matrix component on which the cells which produce the tissue are seeded.

In some embodiments, in vitro-prepared dermal tissues are provided. The term “in vitro-prepared dermal tissue”, as used herein, refers to a tissue that resembles in whole, or in part, skin, or a layer of skin (e.g. dermis) that has been synthesized in a controlled environment outside of a living organism. In some embodiments, the in vitro-prepared dermal tissue resembles in whole, or in part, only the dermis of skin. In some embodiments, the in vitro-prepared tissue is a tissue (e.g., a fibrotic tissue) that resembles a component of skin below the epidermal layer.

DERMAGRAFT (Interactive Wound Dressing) is an example of a commercially available in vitro-prepared dermal tissue and may be used in the methods disclosed herein (Gail K. Naughton, Dermal Equivalents, Chapter 63, Principles of Tissue Engineering, Second Edition, Academic Press, Copyright 2000; and Jonathan N. Mansbridge, Growth factors secreted by fibroblasts: role in healing diabetic foot ulcers, Diabetes, Obesity and Metabolism, 1, 1999, 265-279, the contents of which relating to DERMAGRAFT are incorporated herein by reference). DERMAGRAFT is a cryopreserved human fibroblast-derived dermal substitute that is composed of fibroblasts, extracellular matrix, and a bioabsorbable scaffold. DERMAGRAFT is manufactured from human fibroblast cells derived from newborn foreskin tissue. During the manufacturing process, the human fibroblasts are seeded onto a bioabsorbable polyglactin mesh scaffold. The fibroblasts proliferate to fill the interstices of this scaffold and secrete various factors, including collagen, matrix proteins, growth factors, and cytokines. This creates a three-dimensional human dermal substitute that contains metabolically active, living cells and that is rich in human matrix proteins including Type I collagen (typically 80% total protein by weight) and various proteoglycans. DERMAGRAFT does not contain macrophages, lymphocytes, blood vessels, or hair follicles. The fibroblasts that exist in DERMAGRAFT remain viable (i.e., alive) after thawing. The human fibroblast cells are from a qualified cell bank, which has been extensively tested for animal viruses, retroviruses, cell morphology, karyology, isoenzymes, and tumorigenicity. Reagents used in the manufacture of DERMAGRAFT are tested and found free from viruses, retroviruses, endotoxins, and mycoplasma before use. DERMAGRAFT is manufactured with sterile components under aseptic conditions within the final package. Prior to release for use, each lot of DERMAGRAFT is evaluated by USP Sterility (14-day), endotoxin, and mycoplasma tests. DERMAGRAFT is supplied frozen in a clear bag containing one piece of approximately 2 in by 3 in (5 cm by 7.5 cm) for a single-use application. The product is stored at −75° C.±10° C. (−103° F.±8° F.) and is delivered to customers in shipping containers packed with dry ice. DERMAGRAFT has been approved as a Class III medical device by the Food and Drug Administration (FDA PMA P000036) as a therapy for the treatment of full-thickness non-healing diabetic foot ulcers and is manufactured and marketed by Advanced BioHealing Inc. In addition, DERMAGRAFT is currently under investigation as a Class III medical device for the treatment of Venus Leg Ulcers (VLU) under IDE G090056.

In vitro-prepared tissues are not limited to any particular shape, size or dimension. Often, in vitro-prepared tissues have an aspect ratio similar to that of a sheet or other thin object, e.g., a disc. For example, a tissue that is a rectangular-shaped sheet typically has a thickness (z-axis) that is substantially smaller than its length (x-axis) and than its width (y-axis). The ratio of the longest dimension to shortest dimension (the aspect ratio) of in vitro-prepared tissues may be about 10000 to 1, about 5000 to 1, about 2000 to 1, about 1000 to 1, about 500 to 1, about 200 to 1, about 100 to 1 or about 10 to 1. The shortest dimension (thickness) may be in a range of about 0.01 mm to about 0.05 mm, about 0.05 mm to about 0.1 mm, about 0.1 mm to about 0.5 mm, about 0.5 mm to about 1 mm or about 1 mm to about 5 mm. The shortest dimension (thickness) may be about 0.01 mm, about 0.05 mm, about 0.1 mm, about 0.2 mm, about 0.5 mm, about 1 mm, about 5 mm or more. The longest dimension (e.g., length, width, diameter) may be in a range of about 0.1 cm to about 0.5 cm, about 0.5 cm to about 1 cm, about 1 cm to about 5 cm, about 5 cm to about 10 cm, or about 10 cm to about 50 cm. The longest dimension (e.g., length, width, diameter) may be about 0.1 cm, about 0.5 cm, about 1 cm, about 2.5 cm, about 5 cm, about 7.5 cm, about 10 cm, about 50 cm or more.

Methods for producing in vitro-prepared tissues, e.g., in vitro-prepared tissues, are well known in the art. In vitro-prepared tissues may be produced by seeding fibroblasts (e.g., tendon fibroblasts, ligament fibroblasts, mucosal fibroblasts, dermal fibroblast (e.g., human foreskin fibroblasts)) on a scaffold formed into a desired three-dimensional structure that mimics the shape and dimensions of a layer of a tissue of interest (e.g., a scaffold that is in the form of a sheet to mimic skin). However, methods for producing in vitro-prepared tissues without the use of a three-dimensional scaffold are also known and may be used to produce in vitro-prepared tissues. (See, Pouyani T, et al., De novo synthesis of human dermis in vitro in the absence of a three-dimensional scaffold. In vitro Cell Dev Biol Anim. 2009 September; 45(8):430-41.) Further exemplary methods for producing in vitro-prepared tissues are disclosed in Gail K. Naughton, Dermal Equivalents, Chapter 63, Principles of Tissue Engineering, Second Edition, Academic Press, Copyright 2000; U.S. Pat. No. 5,443,950; U.S. Pat. No. 5,266,480; U.S. Pat. No. 5,032,508; U.S. Pat. No. 4,963,489; and U.S. Pat. No. 4,472,096, the contents of which regarding preparing in vitro-prepared tissues are incorporated herein by reference. The methods often involve seeding cells onto a scaffold capable of supporting three-dimensional tissue formation and recapitulating important aspects of the in vivo environment, such as, for example, the formation of cell-cell and cell-matrix contacts in three-dimensions. Typically, cells are seeded in vitro onto a scaffold which has been formed into a three-dimensional structure (e.g., a mesh). The cells adhere to the scaffold in three-dimensions and, under the appropriate culture conditions, synthesize extracellular matrix components, including matrix proteins and glycosaminoglycans, and growth factors that further stimulate the tissue production. Eventually, the tissue substantially envelops the scaffold material and extracellular matrix occupies much of the interstitial spaces surrounding the scaffold material and cells.

Scaffolds may be of any appropriate material provided that is a biocompatible material. As used herein, a “biocompatible material” refers to a material that is suitable for an intended function (e.g., extracellular matrix production) of the cells seeded thereon and that does not induce undesirable effects (e.g., an undesirable immune response) in a subject. Examples of scaffold materials include, but are not limited to, polyamides, polyesters, polystyrenes, polypropylenes, polyacrylates, polyvinyl compounds (e.g., polyvinylchloride), polycarbonate (PVC), polytetrafluorethylene (PTFE), thermanox (TPX), nitrocellulose, cotton, cat gut sutures, cellulose, gelatin, and dextran. The scaffold material may be biodegradable or non-biodegradable. The scaffold may be natural or synthetic.

Examples of natural materials include proteinaceous materials, such as collagen or fibrin, and polysaccharidic materials, such as chitosan or glycosaminoglycans (GAGs). Other examples include resorbable silk containing polymers, such as silk fibroin. Commonly used synthetic materials include polyglycolic acid (PGA), polylactic acid (PLA), polycaprolactone (PCL) and combinations thereof (e.g., polyglactin). These materials readily degrade in vivo forming metabolites which are easily removed from the body. In addition, polymers offer distinct advantages in that their sterilizability and relative biocompatibility have been well documented. In addition, their degradation rates can be tailored to match that of new tissue formation. For example, PLA is more hydrophobic and less crystalline than PGA and degrades at a slower rate, and thus, the degradation rate of a copolymer comprising the two can be easily controlled by altering the ratio of PLA to PGA in the formulation.

Certain materials, such as nylon, polystyrene, etc., are poor scaffolds for cellular attachment. When materials such as these are used as the scaffold, it is often useful to pre-treat the scaffold prior to seeding cells in order to enhance the attachment of the cells to the scaffold. For example, prior to cell seeding, a scaffold may be treated with an acid (e.g., acetic acid, sulfuric acid) and subsequently incubated with a substance (e.g., polylysine, serum protein, collagen) that adsorbs to the acid-treated scaffold and promotes cell attachment.

A scaffold is typically formed into a three-dimensional structure (e.g., a fiber mesh). The matrix may be formed by any appropriate method known in art. Often the method selected will depend on the scaffold material used. Examples of methods that create porous scaffolds that facilitate cell seeding and migration, include, but are not limited to, polymer knitting, fiber bonding, solvent casting/particulate leaching, gas foaming, emulsification/freeze-drying and phase separation (Mikos A G and Temenoff J S, Electronic Journal of Biotechnology pp. 1-6, Vol. 3 No. 2, Issue of Aug. 15, 2000). Computer-aided design and manufacturing technologies are particularly useful, in some instances, for producing matrices having controlled pore sizes. Here, a three-dimensional matrix is designed using computer-aided design software, then the scaffold is produced by printing of polymer powders or by fused deposition modeling of a polymer melt (Jennifer Elisseeff; Peter X. Ma (2005). Scaffolding In Tissue Engineering. Boca Raton: CRC. ISBN 1-57444-521-9). Other appropriate methods for forming three-dimensional matrices are disclosed herein and will be apparent to the skilled artisan.

In vitro-prepared tissue may be cryopreserved. The cryopreservation may be performed under conditions that maintain or do not maintain cell viability. If maintenance of cell viability following cryopreservation is desired, the in vitro-prepared tissue is typically stored in the presence of a cryopreservation agent, such as dimethyl sulfoxide, serum or other similar agent. If maintenance of cell viability is not required, or desired, a cryopreservation agent is not typically used. Usually, the in vitro-prepared tissue is thawed completely prior to implanting the tissue. Thawing is typically completed within about 2 minutes, but not usually more that 3 minutes, following removal of the tissue from cryopreservation. Thawing is typically performed at an average temperature of 34° C. to 37° C.

### Methods for Tissue Regeneration

Methods for regenerating bone-tendon interfaces in a subject are provided herein. The methods may involve implanting an implantable article comprising an in vitro-prepared tissue between a detached tendon and a bone in a subject. As used herein, the term “detached tendon” refers to a tendon that has been completely, or partially, separated from a bone by surgical means (e.g., by cutting) or non-surgical means (e.g., by traumatic injury or by chronic injury and tissue degradation). A variety of conditions may result in damage that necessitates regeneration of a bone-tendon interface. Conditions resulting in such damage may be traumatic, degenerative, endocrine, metabolic, or inflammatory in nature. For example, stress-related injuries (e.g., due to overuse or excessive muscle or tendon strain) may involve a tear or partial tear at or near a bone-tendon interface. Inflammatory and non-inflammatory conditions (e.g., tendinopathies, enthesopathies) may also lead to damage at or near a bone-tendon interface. Certain bone fractures may also require regeneration of a bone-tendon interface to restore joint function.

As used herein, the term “subject” refers to a mammal, including, but not limited to, a dog, cat, horse, cow, pig, sheep, goat, chicken, rodent, or primate. Subjects include house pets (e.g., dogs, cats), agricultural stock animals (e.g., cows, horses, pigs, chickens), laboratory animals (e.g., sheep, mice, rats, rabbits), and zoo animals (e.g., lions, giraffes), but are not so limited. The subject may be of either sex. Preferred subjects are human subjects. The subject may be a pediatric, adult or a geriatric subject.

As used herein, the term “tendon” refers to a fibrous tissue composed of parallel arrays of closely packed collagen fibers that connects muscle to bone. Contractile forces produced by skeletal muscle fibers are transmitted to tendon at a muscle-tendon interface, and they are transmitted from tendon to bone at a bone-tendon interface. When a tendon is partially or completely torn in a way that requires surgery to restore a more fully functional connection between the tendon and bone, all or a portion of an existing bone-tendon interface may be removed, and thus, regeneration of the bone-tendon interface may be required. As used herein, the term “bone-tendon interface” refers to a structure that connects a tendon with a bone. Bone-tendon interfaces typically comprise a tendon portion, a bone portion and an enthesis located at, or near, the tendon insertion site of the bone. An enthesis may consist of a collagenous structure attached directly to bone, or a transitional series of tissue layers extending from the tendon to the bone that may include fibrocartilage and calcified fibrocartilage. While useful for repairing any torn tendon, the methods are particularly useful for repairing tendons of the rotator cuff.

The methods disclosed herein may be used to regenerate bone-tendon interfaces associated with any tendon. The tendon may be selected from: Abductor Digiti Minimi tendon, Abductor Pollicis Brevis tendon, Abductor Pollicis Longus tendon, Adductor Pollicis tendon, Anconeus tendon, Achilles tendon, Biceps Brachii tendon, Brachialis tendon, Brachioradialis tendon, Coracobrachialis tendon, Deltoid tendon, Extensor Carpi Radialis Brevis tendon, Extensor Carpi Radialis Longus tendon, Extensor Carpi Ulnaris tendon, Extensor Digiti Minimi tendon, Extensor Digitorum Communis tendon, Extensor Digitorum Index tendon, Extensor Digitorum Middle tendon, Extensor Digitorum Ring tendon, Extensor Digitorum Small tendon, Extensor Indicis tendon, Extensor Pollicis Brevis tendon, Extensor Pollicis Longus tendon, First Dorsal Interosseous tendon, First Palmar Interosseous tendon, Flexor Carpi Radialis tendon, Flexor Carpi Ulnaris tendon, Flexor Digiti Minimi tendon, Flexor Digitorum Profundus Index tendon, Flexor Digitorum Profundus Middle tendon, Flexor Digitorum Profundus Ring tendon, Flexor Digitorum Profundus Small tendon, Flexor Digitorum Superficialis Index tendon, Flexor Digitorum Superficialis Middle tendon, Flexor Digitorum Superficialis Ring tendon, Flexor Digitorum Superficialis Small tendon, Flexor Digitorum Superficialis tendon, Flexor Pollicis Brevis tendon, Flexor Pollicis Longus tendon, Fourth Dorsal Interosseous tendon, Index Lumbrical tendon, Infraspinatus tendon, Interossei tendon, Latissimus Dorsi tendon, Levator Scapulae tendon, Lumbricals tendon, Middle Lumbrical tendon, Opponens Digiti Minimi tendon, Opponens Pollicis tendon, Palmaris Brevis tendon, Palmaris Longus tendon, Pectoralis Major tendon, Pectoralis Minor tendon, Pronator Quadratus tendon, Pronator Teres tendon, Rhomboid Major tendon, Rhomboid Minor tendon, Ring Lumbrical tendon, Second Dorsal Interosseous tendon, Second Palmar Interosseous tendon, Serratus Anterior tendon, Small Lumbrical tendon, Subclavius tendon, Subscapularis tendon, Supinator tendon, Supraspinatus tendon, Teres Major tendon, Teres Minor tendon, Third Dorsal Interosseous tendon, Third Palmar Interosseous tendon, Trapezius tendon, and Triceps Brachii tendon.

The methods are particularly useful for repairing damage to tendons of the rotator cuff, which include subscapularis tendon, supraspinatus tendon, infraspinatus tendon, and teres minor tendon. Thus, the bone of the bone-tendon interface is often on the humerus. However, the bone-tendon interface may be on the scapula in some cases. The tendon insertion site is typically on the humeral head of the humerus. For example, the tendon insertion site may be on the lesser tuberosity on the anterior aspect of the humeral head or at the greater tuberosity of the humeral head, depending on the tendon.

Implantable articles and methods of the invention are not limited to use in the repair of bone-tendon interfaces, and may be used to repair other types of connective tissue damage. For example, the implantable articles, and methods of use thereof, may be used to repair ligament damage (e.g., repair bone-ligament interfaces). As used herein, the term “ligament” refers to a fibrous tissue composed of parallel arrays of closely packed collagen fibers that connects bone to bone. The methods disclosed herein may be used to regenerate bone-ligament interfaces associated with any ligament. For example, the ligament may be selected from: anterior cruciate ligament (ACL); lateral collateral ligament (LCL); posterior cruciate ligament (PCL); medial collateral ligament (MCL); cranial cruciate ligament (CrCL); caudal cruciate ligament (CaCL); cricothyroid ligament; periodontal ligament; suspensory ligament of the lens; anterior sacroiliac ligament; posterior sacroiliac ligament; sacrotuberous ligament; sacrospinous ligament; inferior pubic ligament; superior pubic ligament; palmar radiocarpal ligament; dorsal radiocarpal ligament; ulnar collateral ligament; and radial collateral ligament. Thus, devices and methods disclosed herein for tendon repair may be similarly used for ligament repair.

### Implantation Methods

The skilled artisan will appreciate that a variety of approaches may be used for implanting in vitro-prepared tissue, e.g., to facilitate attachment of a tendon or ligament to a bone in a subject. Aspects of the invention are based on improved methods for implanting an in vitro-prepared tissue between a detached tendon or detached ligament and a bone in a subject. Aspects of the invention are particularly useful for minimally invasive techniques. It should be appreciated that the implantation methods disclosed herein for tendon repair may be similarly used for ligament repair.

A method of the invention for implanting an in vitro-prepared tissue between a detached tendon and a bone in a subject typically comprises obtaining an implantable article and positioning the implantable article such that a suture joining the detached tendon with the bone fits within the slit of the implantable article. Thus, the methods also often comprise attaching a first portion of at least one suture to a detached tendon and attaching a second portion of the at least one suture to a bone. The methods further comprise tensioning the suture so that the implantable article is mechanically compressed between the detached tendon and the bone at the tendon insertion site. Attaching and positioning may performed during an open surgery on the subject or during a minimally invasive surgery on the subject. Any implantable article of the invention may be used.

In a typical case involving tendon repair, prior to positioning the implantable article on the tendon insertion site, the site is often cleaned of damaged or residual soft tissue, and/or decorticated. Sometimes, the site is not cleaned of damaged or residual soft tissue, and the implantable article is positioned directly over the area of the tendon insertion site. In some cases, the implantable article may be positioned between the detached tendon and residual tendon material, or an enthesis, that remains associated with the bone.

For minimally invasive surgeries the methods typically comprise percutaneously inserting a cannula for accessing the implantation site (e.g., the site between the detached tendon and the bone in a subject). Thus, the methods may comprise obtaining an introducer device of the invention, wherein the implantable article is disposed within the distal internal cavity of the device, inserting the device into the cannula, and causing a plunger of the device to move to an extended position thereby ejecting the implantable article between the detached tendon and the bone such that the at least one suture fits within the slit of the implantable article.

The implantable article is typically held in place between the detached tendon and bone by compression forces exerted between the detached tendon and bone by a suture. The implantable article may also be sutured directly to the tendon and/or bone. The detached tendon may be fastened to bone by any appropriate methods known in the art, provided that the implantable article is positioned between the tendon and bone, and if need be, mechanical compression forces are exerted on the implantable article to hold it in place. Typically, a first portion of at least one suture is attached to the detached tendon and a second portion of the at least one suture is attached to the bone. Any appropriate number of sutures may be used to fasten the detached tendon to the bone. Multiple implantable articles may be implanted at a surgical site, each sliding onto a different (or the same) suture. Once the suture is connected to the tendon and to the bone, the implantable article may be positioned between the tendon and bone and fixed in place by tensioning the suture so that the implantable article is compressed between the detached tendon and the bone at the tendon insertion site of the bone. An implantable article may also be attached to a portion of the tendon not opposed to the bone, with or without an attachment to bone.

The skilled artisan will appreciate that variety of other approaches may be used for implanting in vitro-prepared tissue(s). The methods may be used, for example, to facilitate attachment of a tendon to a bone in a subject. In some embodiments, all or a portion of an in vitro-prepared tissue is wrapped over the end of the tendon. In other embodiments, all or a portion of an in vitro-prepared tissue is wrapped over the end of the bone, or a portion of the bone that comprises the tendon insertion site. In still other embodiments, all or a portion of an in vitro-prepared tissue is wrapped over the end of the tendon and all or a portion of another in vitro-prepared tissue is wrapped over the end of the bone, or a portion of the bone comprising the tendon insertion site. In some embodiments, a portion of an in vitro-prepared tissue is wrapped over the end of the tendon and another portion of the same in vitro-prepared tissue is wrapped over the end of the bone, or a portion of the bone comprising the tendon insertion site.

The in vitro-prepared tissue is often positioned on the bone such that it substantially covers the tendon insertion site. The in vitro-prepared tissue may be implanted as a single layer or as multiple layers. Multiple layers may be formed by implanting separately prepared tissues, by implanting stacked layers of tissue sections that have been cut from a larger tissue, or by folding a thin tissue into layers. For example, when the in vitro-prepared tissue is a sheet, layers may be formed by folding the sheet to form at least two layers and positioning the folded sheet on the tendon insertion site. Sometimes, the in vitro-prepared tissue is cut to a size that is sufficient to cover a tendon insertion site. For example, a sheet may be cut to a size slightly larger than the tendon insertion site prior to positioning the tissue on the tendon insertion site.

The in vitro-prepared tissue is typically held in place between the detached tendon and bone by compression forces exerted between the detached tendon and bone by suture materials. However, the in vitro-prepared tissue may also be sutured directly to the tendon and/or bone. The detached tendon may be fastened to bone by any appropriate methods known in the art, provided that the in vitro-prepared tissue is positioned between the tendon and bone, and if need be, mechanical compression forces are exerted on the in vitro-prepared tissue to hold it in place. Typically, a first portion of at least one suture is attached to the detached tendon and a second portion of the at least one suture is attached to the bone. Any appropriate number of sutures may be used to fasten the detached tendon to the bone. Once the suture is connected to the tendon and to the bone, the in vitro-prepared tissue may be fixed in place between the tendon and bone by tensioning the suture so that the tissue is compressed between the detached tendon and the bone at the tendon insertion site of the bone. The foregoing methods may be employed in open, mini-open or arthroscopic surgical procedures.

A variety of methods are known in the art and may be used for attaching sutures to tendons or ligaments, including, for example, use of a simple stitch, a mattress stitch, a Kleinert technique, a modified Kessler technique, a Kessler technique, a locking loop, a Mason-Allen technique and a modified Mason-Allen technique. In some cases, the suture attachment to tendon is augmented with a bioabsorbable membrane. Similarly, a variety of methods are known in the art and may be used for attaching sutures to bone, including, for example, cortical graft, metallic brush, double transosseous fixation, single transosseous fixation, the use of Mitek G II anchors, or similar bone anchors, and by membrane augmentation. Examples of methods for attaching sutures to tendon and bone are disclosed in Gerber C, et al., Mechanical Strength of Repairs of the Rotator Cuff, J. Bone Joint Surgery 1994 76-B:371-80. Other examples will be apparent to the skilled artisan.

### Introducer Devices

Devices for introducing an implantable article into a subject are also provided. The devices may be used for repairing detached tendons and detached ligaments according to the methods disclosed herein. The devices typically comprise an elongated sheath having an distal opening, a distal internal cavity and a proximal internal cavity. The distal internal cavity is shaped to receive an implantable article through the distal opening. A plunger is typically provided to facilitate ejection of the implantable article at the implantation site. The plunger has a distal end and a proximal end. The distal end is adapted to interface with the implantable article in the distal internal cavity. The proximal end is fitted within the proximal internal cavity such that it can move axially within the sheath. An actuator is typically provided for moving the plunger axially within the sheath between a retracted position and an extended position. The actuator is coupled with the plunger at the proximal end. Movement of the plunger to the retracted position permits the implantable article to be received in the distal internal cavity, and movement of the plunger to the extended position causes the implantable article to be ejected from the distal internal cavity.

The devices may be used with any implantable article of the invention. During a minimally invasive surgery, the device is passed through a cannula for accessing an implant site. The cannula has an elongated body defining a passage for receiving the device. Ejection of the implantable article is typically performed such that the suture slides into the slit of the implantable article as it is ejected from the device. During a minimally invasive surgery this process may be monitored by one or more cameras at the surgical site. To further facilitate sliding of the suture into the slit, the sheath itself may form a distal slit extending axially from the distal opening through at least a portion of the distal internal cavity. The implantable article may be loaded into the device such that the slit of the implantable article aligns with the slit of the sheath. In this case, the suture may be slid into the slit of the device and then the implantable article may be ejected as the device is pulled away from the surgical site, leaving behind the implantable article lodged on the suture.

### Methods for Evaluating Regeneration

Depending on a variety of factors, including, for example, the location of a bone-tendon interface, the severity of the damage, the age of the subject, the activity level of the subject, the time required for regeneration of the bone-tendon interface will vary. Peak regeneration may occur at about 2 weeks, about 4 weeks, about 8 weeks, about 9 weeks, about 10 weeks, about 11 weeks, about 12 weeks, about 16 weeks, about 20 weeks, about 24 weeks, about 52 weeks or more following implantation. Peak regeneration may occur within a range of about 2 weeks to about 4 weeks, about 4 weeks to about 8 weeks, about 8 weeks to about 12 weeks, about 12 weeks to about 16 weeks, about 16 weeks to about 20 weeks, about 20 weeks to about 24 weeks, or 24 weeks to 52 weeks, or more following implantation, in some cases.

In addition, peak regeneration may be a complete regeneration or a partial regeneration. Partial regeneration may be 10% to 20%, 20% to 30%, 30% to 40%, or 40% to 50%. Partial regeneration may provide a benefit to the subject (patient). Peak regeneration may be about 50%, about 60%, about 70%, about 80%, about 90%, about 95%, about 99% or more. The extent of regeneration may be assessed by any of a variety of parameters, including, for example, molecular composition, mechanical strength, histological appearance. For example, the molecular composition (e.g., collagen content, growth factor content) of the regenerated bone-tendon interface may be determined (e.g., from a biopsy or from sample obtained during an autopsy) and compared to a reference indicative of the molecular composition of a normal bone-tendon interface. The mechanical strength (e.g., elasticity, stiffness, breaking strength) of the regenerated bone-tendon or bone-ligament interface may be determined and compared to a reference indicative of the mechanical strength of a normal bone-tendon interface. The histological appearance (e.g., alignment of collagen fibers, cellularity) of the regenerated bone-tendon interface may be determined and compared to a reference indicative of the histological appearance of a normal bone-tendon interface.

In a clinical setting, non-destructive parameters are particularly useful for evaluating the extent of regeneration. For example, the extent of regeneration may be assessed by imaging (e.g., by MRI imaging, ultrasound radiography). Using imaging, the structural appearance of the regenerated bone-tendon or bone-ligament interface may be determined and compared to a reference image showing the appearance of a normal bone-tendon or bone-ligament interface. Often the normal bone-tendon interface, for example, can be assessed in parallel by imaging a bone-tendon interface in a joint contralateral to the joint in which the regenerated bone-tendon interface resides. The extent of regeneration may also be assessed non-destructively by measuring kinesthetic and kinetic parameters in the subject. For example, the range of motion of the joint comprising the regenerated bone-tendon may be determined and compared with an appropriate reference (e.g., the range of motion of a contralateral joint, the typical range of motion for a subject of similar age, weight, sex, activity level, occupation, etc.) or the change in range of motion of the same joint over time as compared to pretreatment values. The strength of the joint and pain level associated with movement of the joint may also be parameters useful for assessing the extent of regeneration.

Methods for evaluating regeneration of a bone-tendon interface are also provided herein. The methods are useful, for example, to evaluate the ability of an in vitro-prepared tissue to regenerate a bone-tendon interface. The methods may be used to evaluate new types of in vitro-prepared tissues. The methods may also be used to evaluate the performance of existing in vitro-prepared tissues (e.g., as quality control test). For example, in a manufacturing setting, a sample from a lot of in vitro-prepared tissue may be evaluated according to the methods to assess the quality of the lot.

The methods for evaluating regeneration of a bone-tendon interface in a non-clinical setting typically involve detaching a tendon at a bone-tendon interface of a non-human subject (e.g., a sheep, goat, dog, pig) and implanting an in vitro-prepared tissue between the detached tendon and the bone of the bone-tendon interface in the non-human subject. The tendon may be detached using any appropriate means (e.g., by cutting the tendon completely or partially with a scalpel). The implanting step may be carried out using any of the methods disclosed herein. Furthermore, the extent of regeneration may be determined using any of the methods disclosed herein. Sometimes, it is desirable to determine whether or not cells of the implanted tissue persist in the regenerated bone-tendon interface. This may be accomplished in a variety of ways. For example, when the cells of the implanted tissue are of a species other than the species of the non-human subject. The presence of the cells in the bone-tendon interface may be detected by detecting the presence of a marker indicative of the species from which the cells were derived. The marker could be, for example, a cell surface maker, which could be detected by staining a histological section with an antibody. The marker could be a nucleic acid (e.g., mRNA, genomic DNA) having a sequence indicative of the species from which the cells were derived. Such nucleic acid sequences may be determined using any appropriate method known in the art (e.g., qPCR, Padlock probes, cloning and sequencing, etc.). If cells of an in vitro-prepared tissue comprise an exogenous nucleic acid marker (e.g., a transgene expressing a detectable protein (e.g., a luciferase, peroxidase, or a fluorescent protein)), the expression of the detectable protein, or presence of the transgene, may serve to indicate the presence of the cells in the regenerated bone-tendon interface.

It should be appreciated that the methods for evaluating tendons may be similarly applied to evaluate ligaments.

As used herein, the terms “approximately” or “about” in reference to a number are generally taken to include numbers that fall within a range of 1%, 5%, 10%, 15%, or 20% in either direction (greater than or less than) of the number unless otherwise stated or otherwise evident from the context (except where such number would be less than 0% or exceed 100% of a possible value).

All references described herein are incorporated by reference for the purposes described herein.

Exemplary embodiments of the invention will be described in more detail by the following examples. These embodiments are exemplary of the invention, which one skilled in the art will recognize is not limited to the exemplary embodiments.

### EXAMPLES

### Example 1

### In Vitro-Prepared Tissue

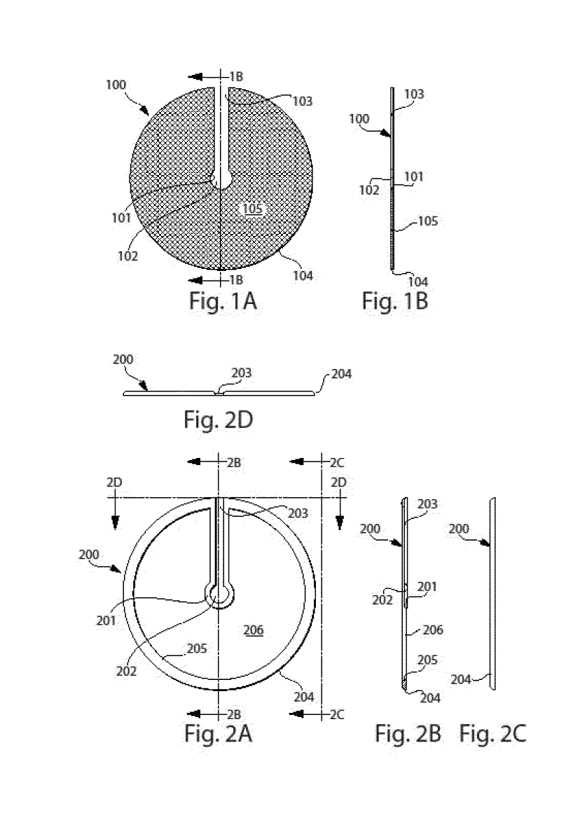

FIG. 1 depicts an embodiment of an in vitro-prepared tissue 100 of the invention. The in vitro-prepared tissue 100 comprises a slit 103, an inner region 101 and a circular shaped passage 102 that adjoins the slit 103 at the inner region 101. The in vitro-prepared tissue 100 also comprises an outer border 104 having a circular shape. The in vitro-prepared tissue 100 of FIG. 1A comprises a scaffold structure 105 made of a biodegradable, biocompatible material attached to which are fibroblastic cells the synthesize extra-cellular matrix growth factors, glycosaminosglycans and other components that make up the in vitro-prepared tissue. FIG. 1B shows a cross-section of the in vitro-prepared tissue 100 of FIG. 1A.

### Example 2

### Support Structure

FIG. 2 depicts an embodiment of a support structure of the invention. The support structure 200 comprises a slit 203, a circular shaped passage 202 that adjoins the slit 203 at the inner region 201. The support structure 200 comprises an outer border 204 The support structure 200 also comprises an interior border 205 that contours the outer border 204, the slit 203 and the circular passage 202. The support structure 200 also comprises an interior opening 206.

### Example 3

### Implantable Articles

FIG. 3 depicts an embodiment of an implantable article of the invention. The implantable article comprises support structure 200₁, support structure 200₂ and an in vitro-prepared tissue 100. FIG. 3A shows an exploded view of the implantable article, which depicts joining structures 300₁ and 300₂. Joining structures 300₁ and 300₂ are compression clamps that hold together to opposing support structures 200₁₋₂. It should be appreciate that, although two joining structures are shown, any number of joining structures may be used. In some embodiments, 1, 2, 3, 4, 5, 6, 7, 8, 9, 10 or more separate joining structures are used to join together multiple support structures. The joining structures may or may not be evenly spaced around the outer border of the support structure. FIG. 3B shows an exploded side view of an implantable article of the invention comprising support structure 200₁, in vitro-prepared tissue 100 and support structure 200₂. FIG. 3C depicts a side view of an assembled, implantable article of the invention comprising support structure 200₁, in vitro prepared tissue 100, support structure 200₂ and a joining structure 300₁. The joining structure 300₁ is a compression clamp that holds together support structure 200₁ and support structure 200₂ such that the in vitro-prepared tissue 100 is sandwiched between the two support structures 200₁ and 200₂. The invention is not limited to joining structures as depicted in FIG. 3 and other appropriate joining structures may be used to join together multiple support structures in the implantable articles of the invention.

Moreover, the implantable articles of the invention are not limited to those having only a single in vitro-prepared tissue. In some embodiments, multiple in vitro-prepared tissues may be stacked together and sandwiched between support structures. For example, the implantable article may comprise, up to 2, up to 3, up to 4, up to 5, up to 6, up to 7, up to 8, up to 9, up to 10, up to 15, up to 20 or more in vitro-prepared tissues. In addition, the size of the in vitro-prepared tissue may be of greater dimensions such that it overlaps the support structure, with a portion of the tissue being outside of the support structures.

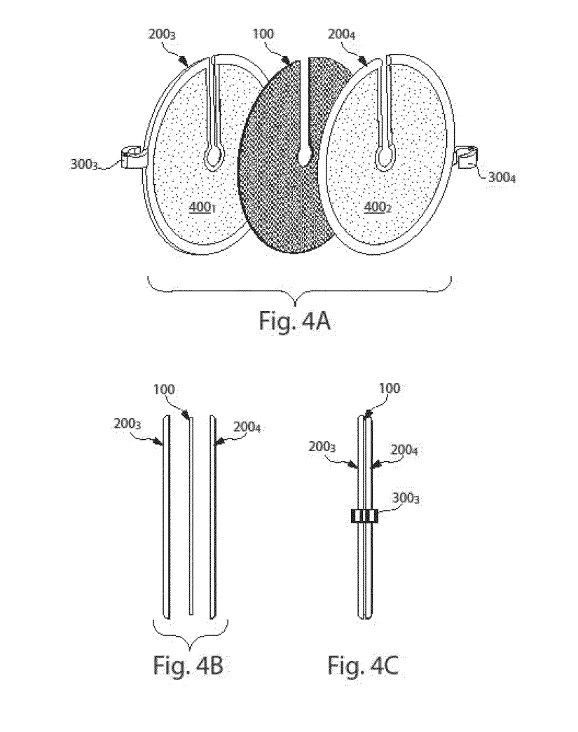

FIG. 4 depicts an embodiment of an implantable article of the invention comprising two support structures 200₃ and 200₄, an in vitro-prepared tissue 100, and two joining structures 300₃ and 300₄. The support structure 200₃ and 200₄ comprise an interior region 400₁ and 400₂, respectively, which are not open but rather comprises a solid or porous structure. Any appropriate solid structure may be used in the interior region of a support structure such as that depicted in FIG. 4. Exemplary interior region materials include any of the scaffolding materials disclosed herein, including, for example, Vicryl, PGA, PLA, PLGA and various other biocompatible and biodegradable materials known in the art. FIG. 4B depicts an exploded view of the implantable article showing the support structure 200₃, in vitro-prepared tissue 100 and support structure 200₄. FIG. 4C depicts an assembled implantable article from a side view showing support structure 200₃, in vitro-prepared tissue 100 and support structure 200₄ held together by joining structure 300₃. In this depiction the joining structure is a compression clamp that holds together the two support structures 200₃₋₄ such that the in vitro-prepared tissue 100 is sandwiched between the two support structures 200₃₋₄. Again, the invention is not limited to the joining structure depicted in FIG. 4C and other appropriate joining structures may be used to hold together or to join together multiple support structures in the implantable articles of the invention. In addition, the size of the in vitro-prepared tissue may be of greater dimensions such that it overlaps the support structure, with a portion of the tissue being outside of the support structures.

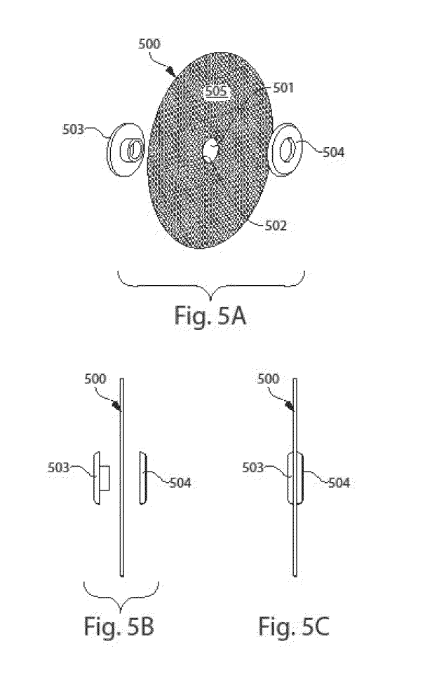

FIG. 5 depicts an alternative implantable article of the invention that comprise an in vitro-prepared tissue 500 comprising a scaffold material 505. Fibroblast or other cells are grown on the scaffold material 505 in order to produce the natural products of the in vitro-prepared tissue. The in vitro-prepared tissue 500 comprises a center passage 501, positioned at an inner region 502. Also depicted in FIG. 5 are two support structures 503 and 504 that snap together at the center passage 501 to provide support for a suture which is passed through the center passage 501. FIG. 5B depicts a side view of the implantable article of FIG. 5A showing support structures 503 and 504 and the in vitro-prepared tissue 500. FIG. 5C shows an assembled view of the implantable article having joining support structures 503 and 504 compressed together at the inner region forming a passage through which a suture may be passed. In this embodiment, the support structure provides support at the center passage 501 in order to prevent tearing of the in vitro-prepared tissue by a suture.

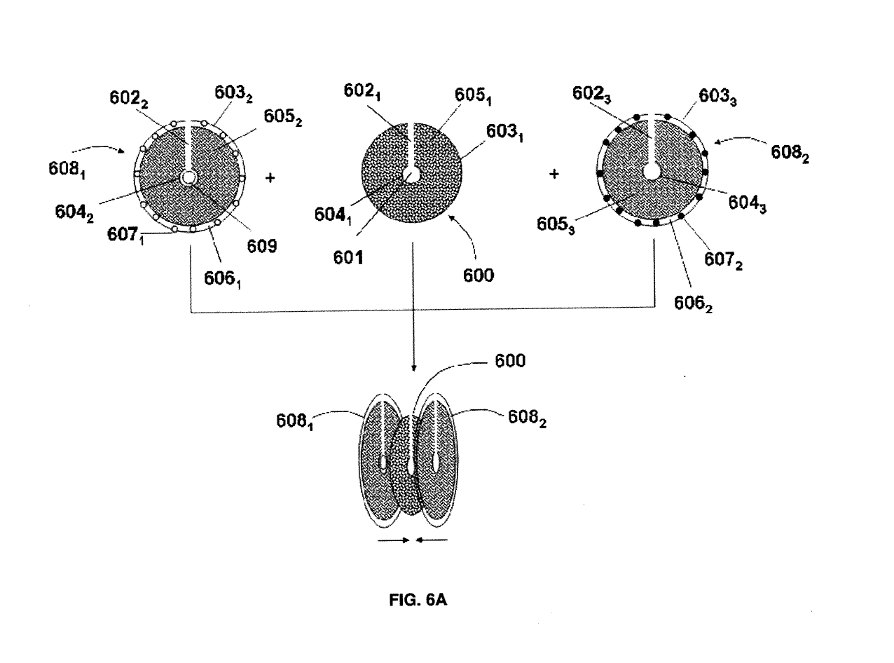

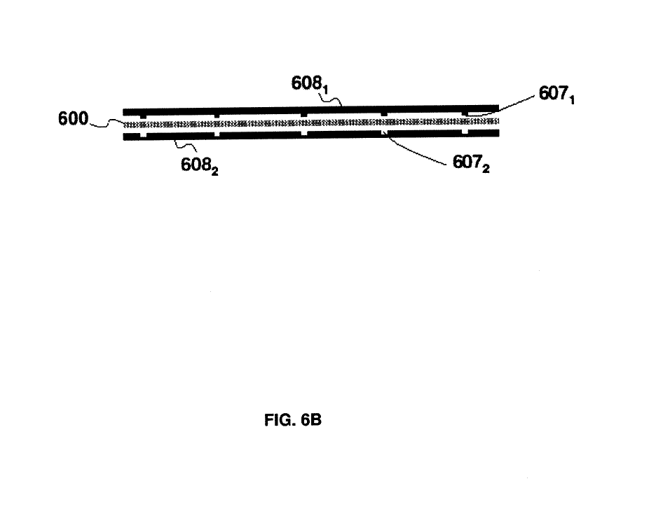

FIG. 6A depicts an embodiment of an implantable article of the invention comprising an in vitro-prepared tissue 600 that comprises a slit 602₁, an outer border 603₁, and an inner region 604₁. The slit 602₁ extends in a straight path from the outer border 603₁ to an inner region 604₁ and has a substantially uniform width. The inner region 604₁ has a circular shaped passage 601 that adjoins with the slit 602₁. The slit 602₁ and passage 601 are shaped to facilitate insertion of a suture through the slit 602₁ and positioning of the suture at the passage 601. In vitro-prepared tissue 600 has a disc shape with a circular outer border 603₁. The in vitro-prepared tissue 600 comprises a mesh portion 605₁ composed of a Vicryl material. An in vitro-prepared tissue 600 is generated on the mesh scaffold 605₁. The in vitro-prepared tissue comprises fibroblasts that synthesize extracellular matrix which adheres/attaches to the scaffold 605₁.

An implantable article of the invention may also include one or more support structures. In the embodiment of FIG. 6A, the implantable article comprises two support structures 608₁₋₂ that provide support for the in vitro-prepared tissue 600. The support structures 608₁₋₂ comprise slits 602₂₋₃ that are aligned with the slit 602₁ of the in vitro-prepared tissue 600. The support structures 608₁₋₂ also have a disc shape. However, in general, support structures 608₁₋₂ need not be shaped identically to the in vitro-prepared tissue 600. The support structures 608₁₋₂ may have slits 602₂₋₃ that permit passage of a suture into their inner region 604₂₋₃. The support structures 608₁₋₂ may comprise an interior mesh portion 605₁₋₂, composed of a vicryl material, and a outer border 606₁₋₂. Though, the interior portion need not be a mesh structure, and may alternatively comprise any type of solid or porous structure. In some embodiments, the support structures 608₁₋₂ do not have an interior mesh portion 605₁₋₂, and instead have an open interior. The support structures 608₁₋₂ can be of a rigid design to give the implantable article a rigid-like shape or can be of a flexible design that facilities deformation of the implantable article and insertion into an arthroscope such that when the implantable article leaves the arthroscope its shape is restored. The material of the outer border 606₁₋₂ portion is often a biodegradable material. Typically, the material of the outer border 606₁₋₂ portion has the same or similar degradation properties as the interior mesh portion 605₂₋₃ Often the in vitro-prepared tissue 600 and the support structures 608₁₋₂ are made from the same material.

The support structures 608₁₋₂ of this embodiment also comprise a plurality of integral joining structures, which are connectors 607₁₋₂ that facilitate snapping together (as depicted in the lower portion of FIG. 6A and in FIG. 6B) such that the in vitro-prepared tissue 600 is sandwiched between the two support structures 608₁₋₂ and the connectors 607₁₋₂ are joined together. This immobilizes the in vitro-prepared tissue 600 between the support structures 608₁₋₂. Immobilization is performed such that the three slits 602₁₋₃ are aligned, thereby facilitating sliding of a suture through the slits 602₁₋₃ into the inner regions 604₁₋₃ of the entire implantable article. The support structures 608₁₋₂ may also comprise a snap 609 at the inner region 604₁₋₂. The snap 609 joins the two support structures 608₁₋₂ at the inner region 604₂₋₃. FIG. 6B depicts a side view of the two support structures 608₁₋₂ sandwiching the in vitro-prepared tissue 600. The connectors 607₁₋₂ are also depicted.

### Example 4

### System for Forming Implantable Articles

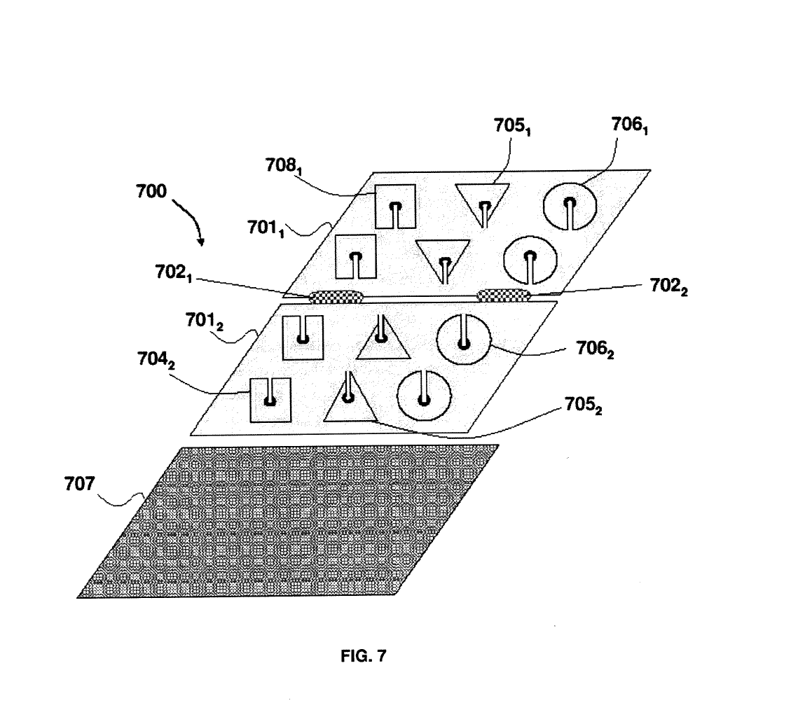

FIG. 7 depicts a system for forming implantable articles having various shapes 700. The system comprises two support structure halves 701₁₋₂ connected by hinges 702₁ and 702₂. The halves may also be separate and not connected by hinges. Each half 701₁₋₂ of the system comprises multiple punch-out support structures of various shapes. Support structures having square cross-sectional shapes are shown 704₁₋₂. Support structures having triangular cross-sectional shapes 705₁₋₂ are shown. Support structures having circular cross-sectional shapes 706₁₋₂ are also shown. A DERMAGRAFT sheet 707 is provided. The DERMAGRAFT sheet 707 comprises a Vicryl mesh scaffold. In order to prepare the implantable articles, the DERMAGRAFT sheet is placed on top of a support structure half 701₂. Support structure half 701₁ is folded on top of the DERMAGRAFT sheet and the individual support structures e.g., 704₁₋₂, 705₁₋₂ and 706₁₋₂ are snapped together such that the DERMAGRAFT sheet is pinched around and compressed between two sides of each support structure component. The individual implantable articles are then released by punching out the support structures e.g., 704₁₋₂, 705₁₋₂ and 706₁₋₂ and cutting or tearing away the DERMAGRAFT.

### Example 5

### System and Method for Minimally Invasive Joint Surgery

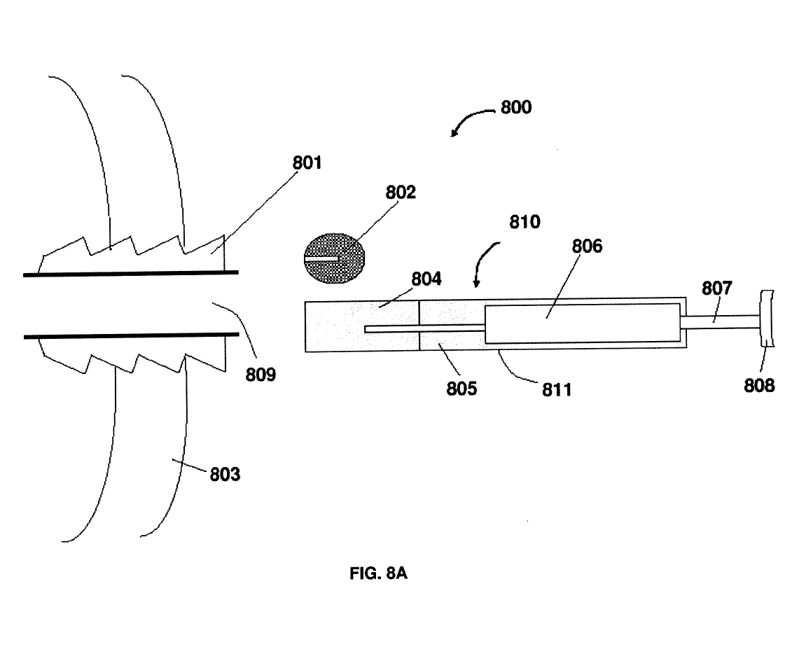

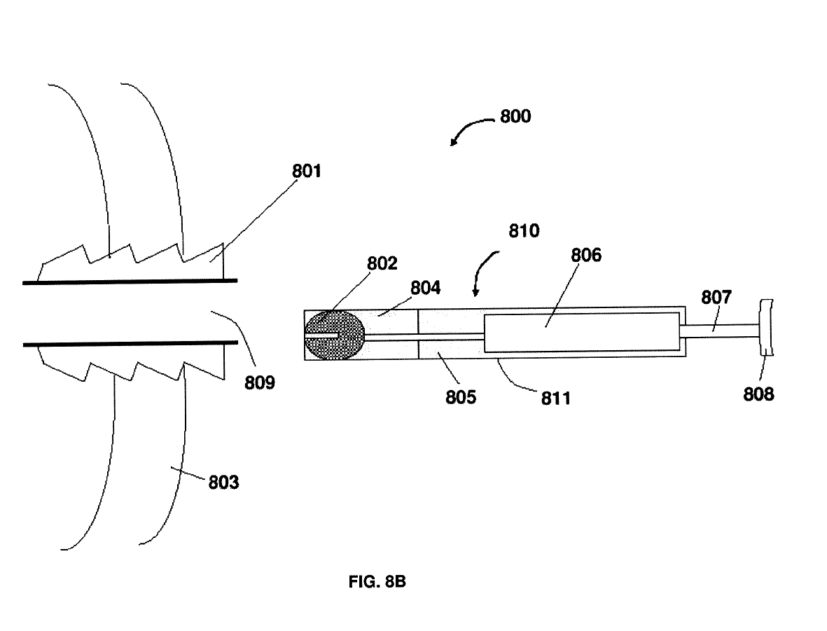

FIG. 8A depicts a system 800 for delivery of an implantable article 802. The system comprises a cannula 801, an implantable article 802 and an introducer device 810 for introducing the implantable article 802 into the cannula 801 to access an implantation site. The introducer device 810 comprises a distal internal cavity 804 that is configured for receiving the implantable article 802 and a proximal internal cavity 805 that permits axial movement of a plunger 806. The internal cavities of the introducer device 810 are defined by a sheath 811. The plunger 806 is also connected to an actuator 807, which in the retracted position is partially external to the sheath 811 and is connected with a handle 808 to facilitate axial movement of the plunger 806 within the device. In the retracted position the introducer device 810 receives the implantable article 802 in the distal internal cavity 804. FIG. 8B shows the implantable article 802 positioned within the distal internal cavity 804 of the introducer device 810.

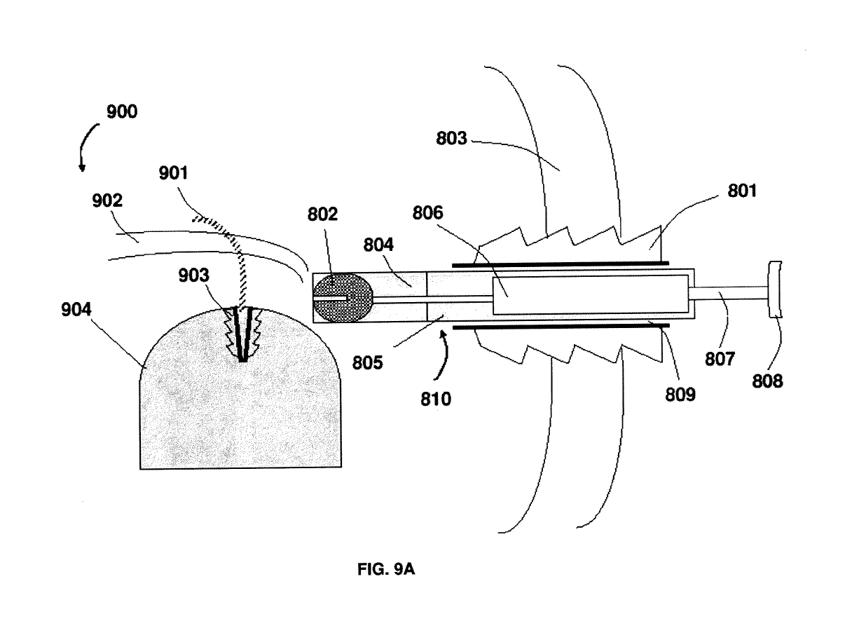

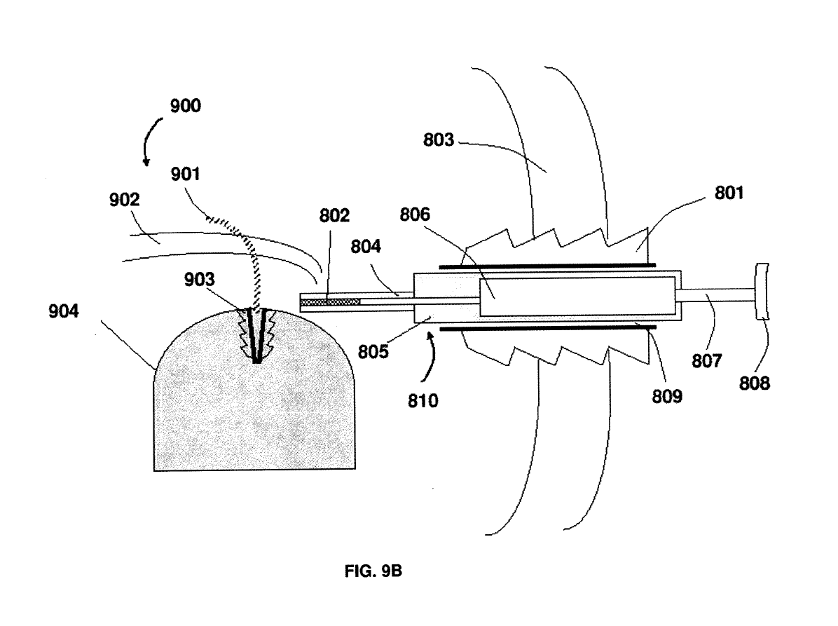

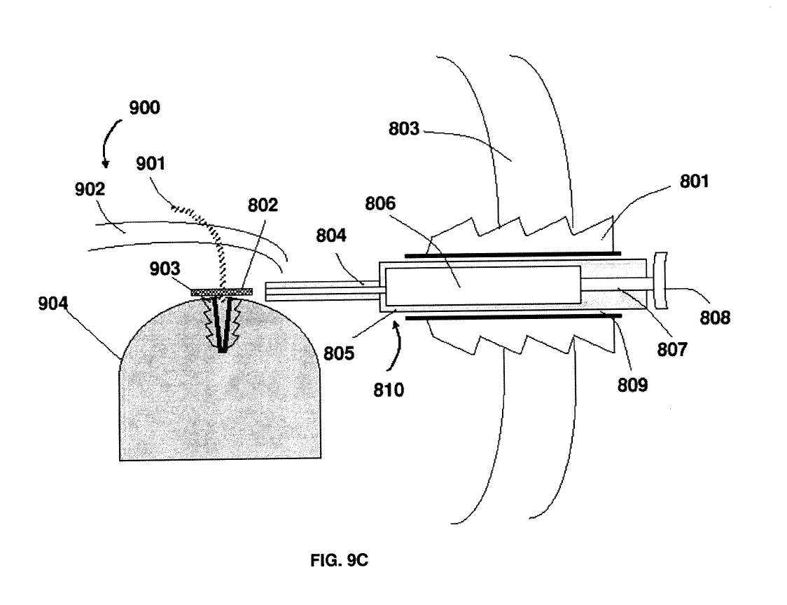

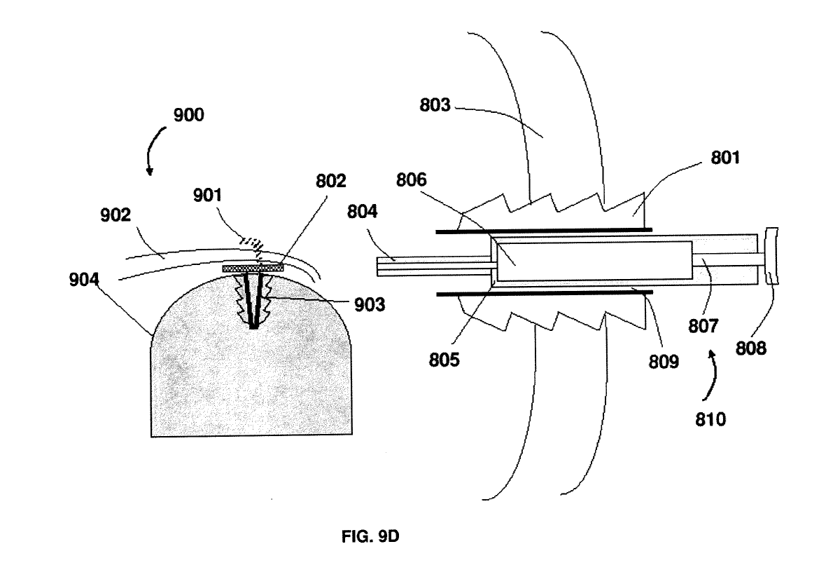

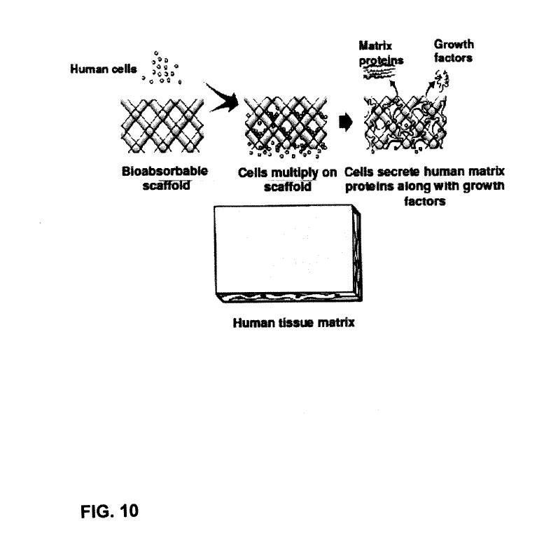

FIG. 9A-D depicts a minimally invasive surgical procedure of the invention using devices and articles of the invention. A surgical site 900 depicting a rotator cuff injury is shown. A detached tendon 902 along with the humeral bone 904 are shown. The detached tendon 902 is connected to the humeral bone 904 via a suture 901 that is connected to the bone via a bone anchor 903. The introducer device 810 is positioned within the lumen 809 of the cannula 801. The implantable article 802 is positioned within the distal internal cavity 804 the introducer device 810 and the plunger 806 is in the fully retracted position.

FIG. 9B shows a cross-section of the introducer device 810 in an alternative orientation and depicts a small internal dimension of the distal internal cavity 804 relative to the proximal internal cavity 805. The internal dimension of the distal internal cavity 804 is fit to accommodate the thickness to the implantable article 802. In FIG. 9B, the disc-shaped implantable article 802 is positioned within the distal internal cavity 804 of the introducer device 810.

FIG. 9C shows the introducer device 810 in an extended position with the actuator 807 present in the proximal internal cavity 805 of the introducer device 810. The implantable article 802, which has been ejected from the distal internal cavity 804 of the introducer device 810, is positioned over the humeral bone 904. The suture 901 is passing through the inner region of the implantable article 802.

FIG. 9D depicts the implantation site 900 where the detached tendon 902 has been brought into position next to the implantable article 802 by tensioning of the suture 901, such that the implantable article 902 is sandwiched between the detached tendon 902 and the humeral bone 904.

### Example 6

### Components and methods for arthroscopic delivery of DERMAGRAFT

DERMAGRAFT is currently manufactured by culturing human dermal fibroblasts on a Vicryl mesh scaffold. The fibroblasts proliferate on the scaffold and generate a collagen-rich extracellular matrix forming a three-dimensional structure. Arthroscopic delivery involves fitting a circular piece of DERMAGRAFT, of approximately 5-10 mm in diameter, within a flexible ring (reinforcement member) composed of a biodegradable material that is compacted and passed down the arthroscope, but which regains its shape once it emerges from the end of the arthroscope. As shown in FIG. 7, the piece of DERMAGRAFT may have alternative shapes and sizes. The material for the ring is bioresorbable such as PLA/PGA. The DERMAGRAFT may be sandwiched between two layers of Vicryl mesh or other bioresorbable material prior to capture with the support structure to help support the DERMAGRAFT in the bioresorbable rings, and to protect it from damage as it is passed down the arthroscope.

The ring is composed of two matched halves that allow for the two sections to be held together, either by snaps or by other joining structures, thereby allowing for the capture of a DERMAGRAFT between them. A central button hole is also present with either a single passage or multiple passages for holding anchoring sutures once the article is delivered to the repair site in the patient. Another feature of the article includes a single slit that is either cut into the DERMAGRAFT directly, that extends from the outer circular ring through the DERMAGRAFT to the central button hole, or a slit that is a component of the ring that is composed of a similar material as the ring (or another biocompatible material) that provides a guide for sliding the suture into the central ring.

The article is designed to allow a surgeon to take advantage of sutures that have been placed into the bone (such as by an anchor) and/or tendon and which are intended to be used to attach the tendon to the bone as a part of the repair process for treating rotator cuff tears. Once placed into the bone or tendon, the disc is slid onto the sutures by way of the slit created in the disc, allowing the sutures to be directed into the central button in the disc. Once the DERMAGRAFT disc is placed, the surgeon secures the product beneath the overlying tendon using the sutures within the slit or additional sutures at the repair site to secure the tendon onto the bone.

The diameter of the DERMAGRAFT disc is 5-10 mm, though other disc diameters may be used. A hemicentric slit is cut from the outer ring to the central button to allow the disc to be slid onto the sutures for positioning the disc between the tendon and the bone and for anchoring the DERMAGRAFT at the tendon-bone surgical site. This technique is designed for the delivery and placement of the DERMAGRAFT disc using arthroscopic procedures. The disc may be used in mini-open and in open surgical procedures where arthroscopic delivery is not necessary. The DERMAGRAFT may or may not be sandwiched between two support structures such as Vicryl.

The DERMAGRAFT disc may alternatively be attached on top of the tendon away from the bone using a similar procedure where the disc is slid onto the sutures that are attached to the tendon using the slit in the disc, and the disc held in place on top of the tendon as the sutures are secured to hold the tendon to the bone.

### Example 7

### Preparation of DERMAGRAFT for Arthroscopic Delivery

Preparation of the DERMAGRAFT for arthroscopic delivery and its attachment to the delivery disc is done by using a punch or other cutting instrument, at the time of the use, to form the specific shape of Dermagraft (in vitro prepared tissue) to be incorporated into the implant, inserting it into at the support structure (a flexible ring, potentially with protective Vicryl mesh layers, central button hole, and the hemicentric slit). Alternatively, the ring and associated structures are preformed and delivered to the surgical suite ready for use. The DERMAGRAFT is then added between the two ring halves at the time of use. The ring may be designed as two halves with one half having small knobs or teeth and the other half having matching holes into which the teeth fit and snap together. Alternatively, both ring halves may have suture holes positioned around their circumference to enable passage of sutures through both holes following placement of the DERMAGRAFT in between the two ring halves. Each half may include one sheet of supporting material/mesh such as Vicryl, to provide additional support to the DERMAGRAFT. The Vicryl mesh is optional, and other protective materials may be used. The DERMAGRAFT may also be placed between the two bioresorbable rings directly. One or both sides of the ring set may include a central button hole positioned centrally to allow for the capture of the suture when placed in the body.

Alternatively, the DERMAGRAFT may be aligned on either side of a single ring, which has suture holes around its circumference. Once the DERMAGRAFT and ring are aligned, they are sutured together through the pre-formed suture holes in the ring.

### Example 8

### Punch Apparatus

A product is provided that includes a knife or divider to create the appropriately shaped form of Dermagraft to be used. The same knife or divider, or a separate knife or divider may also be used to create the slit. The DERMAGRAFT sheet, which is supplied to the end user in a frozen state, is thawed and placed on the sterilized punch apparatus and the units for arthroscopic delivery are generated efficiently and are ready for implantation in the patient intra-operatively.

### Example 9

### Evaluation of an In Vitro-Prepared Dermal Tissue to Accelerate the Healing of Rotator Cuff Injuries in a Sheep Model

A study was conducted to evaluate the biological compatibility of using a xenograft matrix in a sheep model. The study, which included nine (9) animals in total, had three arms evaluating two doses of DERMAGRAFT and control treatment, n=3 per group. The test articles, or no article at all as a control, were placed within the rotator cuff injury site, according to the methods disclosed herein, immediately after the creation of the injury. The animals were sacrificed and tissues were collected at 9 weeks after treatment. The results of this study indicated a lack of an acute inflammatory reaction, lack of long-term rejection, and the survival of the fibroblasts at the bone-tendon interface after absorption of their scaffold.

This study was designed to determine the biocompatibility of DERMAGRAFT and its impact on healing in a sheep model of rotator cuff injury. The results strongly indicate that DERMAGRAFT was well-tolerated with evidence of neither acute nor chronic rejection of the product. The presence of the SRY marker in the sheep tissues indicated that the human fibroblasts contained within DERMAGRAFT survived for an extended period of time, further supporting biocompatibility of the DERMAGRAFT product. Necropsy observations suggested that suturing of the tendon to the bone was not sufficient to prevent tearing of the tendon away from the bone, as demonstrated by tendon retraction in all animals. This action resulted in a robust healing response by the sheep, leading to neo-tendon formation and incorporation with the mature tendon, which may have masked some of the impact of the DERMAGRAFT treatment on healing.

The fact that some of the impact of the DERMAGRAFT treatment on healing may have been masked in this study is not surprising. Large animal models for rotator cuff repair, such as the sheep model used in this study, are limited by tendon retraction. The tendon retraction forms scar tissue between the detached tendon and bone. An issue with scar tissue is that it can be visually, mechanically, and histologically misconstrued as tendon (Derwin K. A. et al., Preclinical Models for Translating Regenerative Medicine Therapies for Rotator Cuff Repair, Tissue Engineering Part B, Volume 15, Number 00, 2009). Thus, differences between tendon and scar tissue can be a challenge to discern, particularly with small sample sizes. The skilled artisan will appreciate, however, that this study achieved its intended goals of assessing biocompatibility of DERMAGRAFT and the survival of the fibroblasts at the bone-tendon interface after absorption of their scaffold.

TABLE 1

| A three cohort study was | | |
|---|---|---|
| conducted as outlined below | | |
| using the sheep rotator | | |
| cuff injury model previously | | |
| described (**). Group # | Treatment | Sheep (n) |
| 1 | Suture only | 3 |
| 2 | Suture + 1 layer of | 3 |
| | Dermagraft | |
| 3 | Suture + 3 layers of | 3 |
| | Dermagraft | |

(**) J. Shoulder and Elbow Soc. 2007; 16(5 Suppl): S158-63

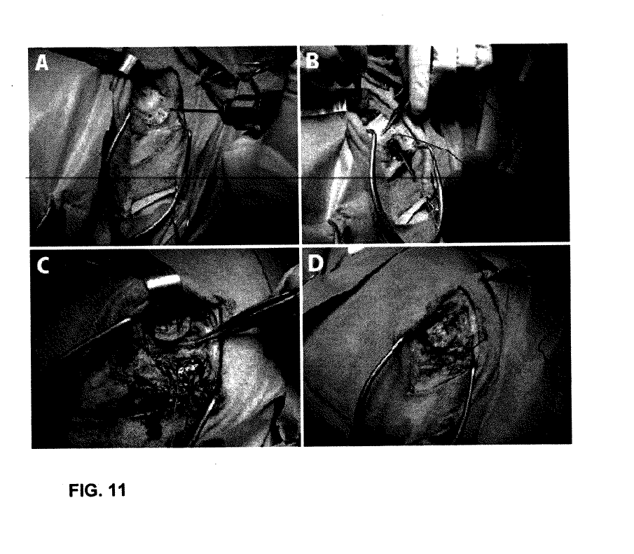

For each animal, on the day of surgery, the infraspinatus tendon was sharply dissected from the head of the humerus. The former insertion site of the tendon (approx. 2 cm×1.5 cm) was decorticated with a Hall orthopedic burr. The infraspinatus tendon was then reattached to the proximal humerus by use of three bone tunnels and sutures (FIG. 11). For the DERMAGRAFT treated animals, 1 or 3 layers of the matrix were “sandwiched” between the infraspinatus tendon and humerus as the sutures were tied. For the suture-only control animals, sutures were used to reattach the tendon. The sheep were euthanized at 9 weeks following treatment and the shoulder tissue harvested.

### Analytical Methods:

Ultrasound: Sagittal and transverse images of the infraspinatus tendon were obtained on live animals at 4 and 9 weeks using an Antares Sonoline Ultrasound machine. A 9 mHz linear transducer was used to view sagittal and transverse images of the infraspinatus tendon.

Gross assessment: Macroscopic analysis of the harvested bone-tendon complex was performed at the time of euthanasia.

Histopathology: Assessment of tissue structure, cellular organization and the presence of abnormal responses were monitored using H&E, toluidine blue and picrosirius-red stained sections of decalcified tissues. Each shoulder was bisected through the infraspinatus and humeral attachment site into cranial and caudal halves. The halves were processed for decalcified histology and embedded in paraffin. Approximately 5-8 micron thick sections were cut and processed for staining which included Hematoxylin-Eosin (HE), picrosirius-red (PR), and toluidine blue (TB).

Persistence of fibroblasts: PCR amplification for the human male Y chromosome marker, SRY, was conducted to determine the presence of the human fibroblasts in harvested rotator cuff tissue. The PCR method was described in Diabetes Obes. Metab. 1999; 1:265-279.

### Study Results

Ultrasound: At 4 weeks, ultrasound analysis showed minimal gap formation between the tendon and bone. The tissue appeared organized, with little evidence of edema or inflammation. Similar findings were observed at 9 weeks. Due to limitations in the model used, no differences were observed using ultrasound between the three treatment groups.

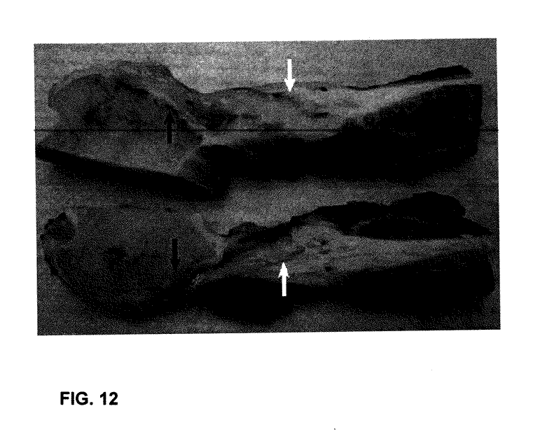

Gross assessment: Visual assessment of the harvested shoulders revealed that the repaired tendon had detached from the bone in all animals and retracted 2-5 cm from the attachment site. FIG. 12 shows a harvested shoulder split down the middle, revealing the level of retraction of the tendon end (white arrows) from the original attachment site (black arrows). The intervening space between the tendon and bone was filled with fibrotic tissue for all treatment groups. There was no evidence of an inflammatory response for any group.

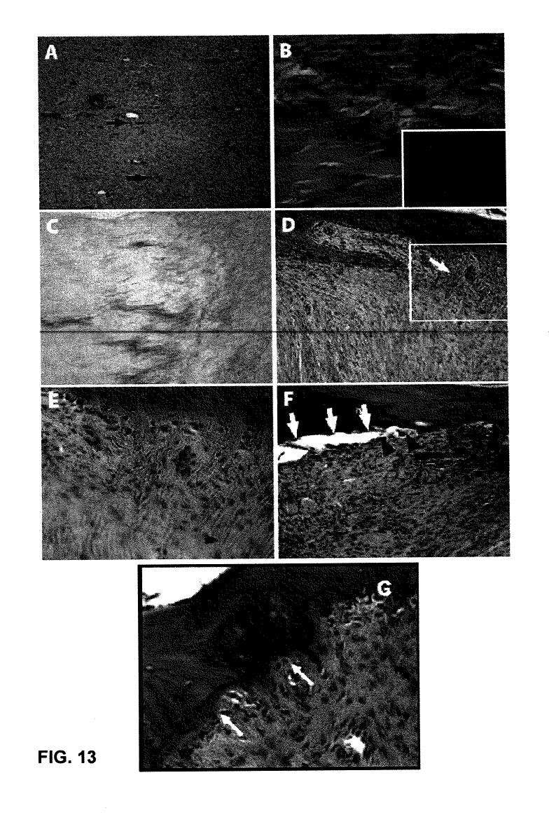

Histopathology: Evaluation of histology sections (FIG. 13) showed that the fibrotic tissue was actively remodeling into “neo-tendon”, which had increased fibroblast cellularity (FIG. 13A). The neo-tendon, which interdigitated and appeared continuous with the cut edge of the retracted mature tendon, had a normal “kinking” pattern resulting in regular periodicity to the structure (FIGS. 13B and 13C). The length of the periodicity was approximately one-half to one-third that of mature tendon. The neo-tendon also had an increased number of blood vessels that were oriented parallel to the longitudinal plane of the tendon. At the bone-tendon interface there were areas of well defined Sharpeys fibers (arrows) anchoring the neo-tendon to the bone with active bone formation, including the presence of reversion lines and lining osteoblasts (FIGS. 13D, 13E and 13G). Other areas showed clefts/voids with osteoclast resorption (arrows) at the tendon/bone interface (FIG. 13F). There was no evidence of an acute inflammatory response for any of the treatment groups. Additionally, no remnants of the DERMAGRAFT vicryl matrix were observed. Due to limitations in the model used, histology did not reveal any differences among the three treatment groups.

Fibroblast Persistence: Results demonstrated that in some animals the fibroblasts contained within DERMAGRAFT remained present at the wound site at 9 weeks, as determined by the detection of the human male Y chromosome marker, SRY.

TABLE 2
Persistence of fibroblasts, as determined by SRY analysis, in
tissues collected at 9 weeks after placement of DERMAGRAFT.

| Group # | Treatment | Sheep # | SRY Analysis |
|---|---|---|---|
| 1 | Sature only | 1 | − |
| | | 2 | − |
| | | 3 | − |
| 2 | Suture + 1 layer | 4 | + |
| | of Dermagraft | 5 | − |
| | | 6 | + |
| 3 | Suture + 3 layers | 7 | + |
| | of Dermagraft | 8 | − |
| | | 9 | − |

### Detailed Materials and Methods

Test Article Preparation

The test article (DERMAGRAFT) was obtained from commercial supplies of the product. The product was supplied frozen and was thawed immediately before use. The product used in this study was thawed according to the methods disclosed herein.

Reserve Samples

Reserve samples of DERMAGRAFT were tested to confirm final product test specifications. The reserve samples were tested using the following methods: MTT analysis, DNA analysis and Sirius red staining.

Test System

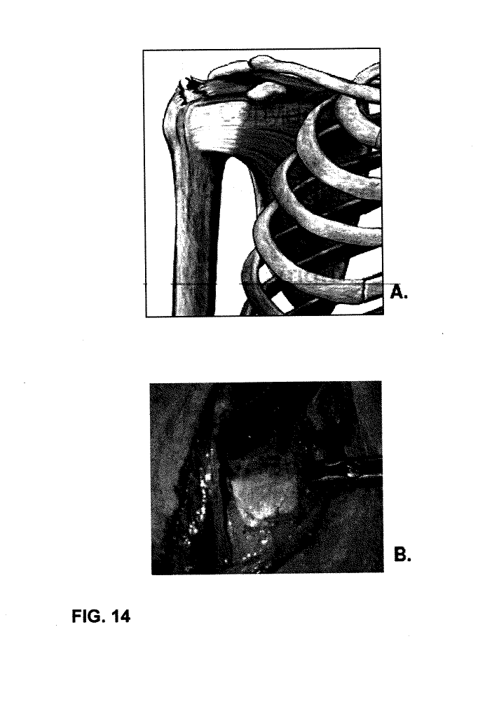

Sheep were used in this study because the size and anatomical structure of their rotator cuff is comparable to that of a human shoulder. For example, the dimension of the human supraspinatus tendon is very close to that of the sheep infraspinatus tendon (Gerber, C., B. Fuchs, et al. (2000). “The results of repair of massive tears of the rotator cuff.” J Bone Joint Surg 82A(4): 505-515). In addition, the sheep is an established model for assessing treatments for rotator cuff injuries. (Turner A. S. Experiences with Sheep as an Animal Model for Shoulder Surgery: Strengths and Shortcomings. J. Shoulder and Elbow Soc. 2007, September-October; 16(5Suppl):5158-63; Rodeo S A, et al., Biologic augmentation of rotator cuff tendon-healing with use of a mixture of osteoinductive growth factors. J Bone Joint Surg Am. 2007 November; 89(11):2485-97; Seeherman H J, et al., rhBMP-12 accelerates healing of rotator cuff repairs in a sheep model J Bone Joint Surg Am. 2008 October; 90(10):2206-19). FIG. 14A shows the human shoulder joint with a torn supraspinatus muscle. FIG. 14B shows an intraoperative picture of the infraspinatus tendon of a sheep shoulder that was used in this study.

TABLE 3
Sheep Model System Details

| | |
|---|---|
| Species | Ovis aries (a.k.a. sheep) |
| Sex/Age/Size | Female, 3+ years of age, 120-180 lbs. |
| Source | Three JP LLC, La Junta, CO. |

Number of Animals

The study design, the number of sheep per group and the number of sheep included for each analytical assessment is listed in Table 3.

Surgical Procedures

Animal Prep and Exposure Surgery

With the sheep in left lateral recumbency, the wool was removed from the right shoulder region. The skin over the right shoulder joint was prepared for aseptic surgery using alternating scrubs of povidone-iodine (Betadine) and alcohol. The joint was draped for aseptic surgery. Under general anesthesia using aseptic conditions, a 6 cm skin incision was made over the shoulder joint. The subcutaneous colli muscle was divided in line with the incision. The deltoid muscle was split along the tendinous division between its acromial and scapular heads. The superficial head and insertion of infraspinatus tendon was isolated and the infraspinatus tendon sharply dissected from the humerus using a scalpel.

Cuff Repair Technique

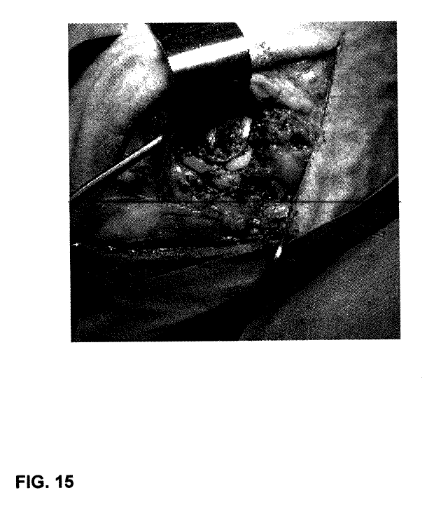

The former insertion site of the infraspinatus tendon on the head of the humerus was decorticated with a Hall orthopaedic burr. A standard area of bone (approximately 2 cm×1.5 cm) was decorticated. The infraspinatus tendon was grasped and reattached to the proximal humerus by use of three bone tunnels and two sutures, using a Mason-Allen pattern (FIG. 15). In the control animals, essentially nothing more was performed (apart from closure). For the DERMAGRAFT test article treated animals, 1 or 3 layers of DERMAGRAFT was “sandwiched” between the infraspinatus tendon and proximal humerus and held in place by passing the sutures through the DERMAGRAFT as they were placed between the bone channels and the tendon. Once the sutures were tied they held the tendon fast to the bone with the DERMAGRAFT in between the tendon and bone (See FIG. 15). The control group had no DERMAGRAFT implanted under the tendon.

A new sheet of DERMAGRAFT was used for each surgery. The thawing procedures for the DERMAGRAFT followed the instructions provided with the commercial product. However, a modification to these instructions was used for the sheep surgeries to provide a quick rinse to assist in the sterilization of the outside of the DERMAGRAFT package for use in the animal surgical suite. It was determined that the addition of isopropyl alcohol rinses of the outside covering of the product, to decrease potential bioburden within the surgical suite, do not impact the quality of the product as determined by measuring the cell viability within the DERMAGRAFT using a validated MTT assay.

Once thawed, the DERMAGRAFT was removed from its packaging material using aseptic techniques, and cut to the appropriate size for placement into the wound site. The thawed DERMAGRAFT was stored in a sterile vessel that was filled with sterile saline, for up to 30 minutes after thawing and prior to placement into the animal. In cases where multiple layers of DERMAGRAFT were implanted, the matrix was folded upon itself to produce the multi-layer matrix. The individual sheets of DERMAGRAFT, either single or multi-layered, were cut to approximately 2 cm in width by 1.5 cm in height. Intra-operative digital photographs were taken after implantation of the DERMAGRAFT and prior to wound closure.

Closure of Wound Site

After attachment of the infraspinatus tendon the surgical site was lavaged with sterile saline. The brachial fascia and subcutaneous tissues were closed as separate layers using 0 Polysorb. Stainless steel staples (Proximate; Ethicon) were used for the skin closure.

Anesthetic Monitoring

The level of anesthetic monitoring and post-operative recovery/care, including frequency and location of post-op monitoring includes: Anesthetic monitoring every 5 minutes: a) EKG, b) arterial blood pressure, c) respiratory rate, d) pulse oximetry, e) capnography, f) end-tidal CO2, g) jaw tone.

TABLE 4
Analgesics

| | When | | | | |
|---|---|---|---|---|---|
| Analgesic drug | Employed | Dose | Route | Frequency | Duration |
| Fentanyl | For all | One 10 mg & | Percutaneous; | Continuous | 3 days. |
| patches | surgeries | one 5 mg patch | applied 24 hrs. | | |
| | | (150 μg/hour) | pre-op. | | |
| Phenylbutazone | For All | 1 gm | Orally | Every 24 | 1 day pre -op., |
| | Surgeries | | | hours | day of surgery |
| | | | | | and 3 more days |
| | | | | | post-op. |
| Morphine | After | 1 mg/kg | Subcutaneously | After | NA |
| | induction | | | induction | |
| | of | | | | |
| | anesthesia | | | | |
| Lidocaine* | For all | 25 μg/kg/min | Intravenous | Constant rate | Lidocaine* |
| | surgeries | | | infusion in | |
| | | | | surgery | |
| Ketamine* | For all | 10 μg/kg/min | Intravenous | Constant rate | Ketamine* |
| | surgeries | | | infusion in | |
| | | | | surgery | |
| Morphine | Bruxism | Constant rate | Intravenous | Post- | Post-operatively |
| | (post op) | infusion 2-4 | | operatively | |
| | | mcg/kg/hour | | | |

All sheep were on isoflurane (typical vaporizer settings, 2 to 2.5%) and were given morphine (1 mg/kg SQ) after induction of anesthesia, before surgery starts. The sheep were also on two CRI infusions. The Ketamine CRI dose was 0.6 mg/kg/hr (1.8 ml Ketamine mixed into 3000 ml bag of Normasol) and the lidocaine CRI dose was 1.2 mg/kg/hr (18 ml 2% lidocaine mixed into 3000 ml bag of Normasol). The crystalloid fluid rate was usually 10 ml/kg/hr. Heart rate and arterial blood pressure were continuously monitored during anesthesia, and if they increased more than 10%, the isoflurane vaporizer setting was turned up accordingly.

Post-Surgical Care

Immediately after surgery, the sheep were transferred from the operating table to a transport vehicle and observed until the swallowing reflex returns. At that point they were extubated and taken to the aluminum stock trailer (a.k.a. a “Sheep Shuttle”) where they were propped in sternal recumbency. At the end of the day, all animals that were operated upon that day were moved to the research barn. The sheep were housed indoors for the duration of the study. While in the barn, they were constantly monitored via a webcam system in the barn. The webcam images are recorded 24/7 on a DVR system. Postoperative analgesia was provided as required.

The antibiotic cefazolin sodium 1 gram was given intravenously to each sheep at induction, midway through the surgery and during closure. Procaine penicillin, 3 million units, was given subcutaneously, once daily to each sheep for 3 days postoperatively.

Euthanasia

At 10 or 20 weeks the sheep were euthanized using Pentobarbital Sodium at a dose of approximately 88 mg/kg delivered via intravenous-jugular injection. Euthanasia was performed according to the guidelines set forth by the AVMA in 2007, “AVMA Guidelines on Euthanasia” June 2007.

Rotator Cuff Tissue Harvesting

Following the death of the sheep the treated shoulders were surgically removed by dissection being careful not to damage the surgical treatment site. In a subset of animals, pre-determined prior to the initiation of the surgical procedure, the contra-lateral untreated shoulder was also harvested at the time of Euthanasia. The explanted shoulders were processed for subsequent analysis.

PCR Analysis for Persistence of Human Fibroblasts

At the time of tissue harvesting, sections of tissues from both control and treated animals were processed for analysis for the persistence of the human dermal fibroblasts at the treated site.

DNA Isolation

Pieces of sheep tissue from the rotator injury sites were collected from those shoulders that underwent histological assessment. A piece of tissue of approximately 2×2 mm in size was harvested from the initial site of implantation adjacent to the tuberocity of the humerus, and at the leading edge of the original tip of the tendon, the location of which depended upon the extent of tendon retraction that occurred subsequent to the surgical procedure. Previous studies had demonstrated that the tendon may retract away from the head of the humerus due to mechanical forces placed on it by the daily activities of the sheep. The tissue samples were either processed immediately for DNA isolation or stored at approximately −70° C. until ready for DNA isolation. DNA was isolated from control and test sheep tissues, as well as from a sample of DERMAGRAFT (positive control), using DNAeasy blood and tissue kits (Qiagen, Valencia, Calif.). These kits used proteinase K digestion followed by column purification to isolate DNA. The resulting purified DNA was stored at −20° C. until analyzed by PCR amplification.

PCR Analysis

Reactions for PCR analysis were prepared in a PCR work station with HEPA filtered air and UV light to destroy DNA/RNA contamination. PCR amplification for the human male Y chromosome marker, SRY, was performed using nested PCR primers in which the first set anneal to SRY residues 544-563 5′-AGTGTGAAACGGGAGAAAAC-3′ (SEQ ID NO: 1) and residues 901-882 5′-TACAACCTGTTGTCCAGTTG-3′ (SEQ ID NO: 2), and the second set anneal to SRY residues 569-588 5′-AGGCAACGTCCAGGATAGAG-3′ (SEQ ID NO: 3) and residues 858-838 5′-GCAATTCTTCGGCAGCATCTT-3′ (SEQ ID NO: 4). These primers had previously been used to amplify the human SRY gene (Refs. 1-3). DNA quality was verified by amplification of the ovine beta actin gene using the following primers 5′-CGCAGACAGGATGCAGAAAGA-3′ (SEQ ID NO: 5) and 5′-GCTGATCCACATCTGCTGGAA-3′ (SEQ ID NO: 6) (Ref. 4). This primer pair spans an intron in the beta actin gene so an amplicon of 148 bp was generated from cDNA transcribed from the mRNA and an amplicon of 260 bp was generated from genomic DNA. PCR amplification of SRY was performed in standard PCR buffers with 0.2 μM primers and 200 μM dNTPs, 2.5 U/reaction of Taq polymerase, and 50-100 ng of template DNA. The amplification format included 30 cycles of amplification with DNA melting at 95° (30 sec.), annealing at 55° (1 min) and extension at 72° (30 sec.) with the first set of primers, followed by 30 rounds of amplification with the second set of primers. Detection of the SRY gene from human male fibroblasts was validated by performing PCR analysis of fibroblast DNA serially diluted in control ovine DNA until only a single copy of the SRY gene was estimated to be present in the input DNA. Separate reactions amplifying the β-actin gene were conducted to verify the quality of the DNA. The amplified PCR reactions were visualized by gel electrophoresis in 2% agarose gels with 0.5 μg/ml of ethidium bromide. The SRY amplicon was 290 bp in length and the beta-actin amplicon was 260 bp in length.

PCR Analysis References
1. Calzolari E, Patracchini P, Palazzi P, Aiello V, Ferlini A, Trasforini G, degli Uberti E, Bernardi F 1993 Characterization of a deleted Y chromosome in a male with Turner stigmata. Clin Genet 43:16-22
2. Petrovic V, Nasioulas S, Chow C W, Voullaire L, Schmidt M, Dahl H 1992 Minute Y chromosome derived marker in a child with gonadoblastoma: cytogenetic and DNA studies. J Med Genet 29:542-546
3. Mansbridge J N, Liu K, Pinney R E, Patch R, Ratcliffe A, Naughton G K 1999 Growth factors secreted by fibroblasts: role in healing diabetic foot ulcers. Diabetes, Obesity & Metabolism 1:265-279
4. Lampo E, Van Poucke M, Hugot K, Hayes H, Van Zeveren A, Peelman L J 2007 Characterization of the genomic region containing the Shadow of Prion Protein (SPRN) gene in sheep. BMC Genomics 8:138

Test Article Administration:

Route of Administration

The DERMAGRAFT was delivered to the surgical site by placing a thawed test article that had been cut to the appropriate size (approximately 2 cm×1.5 cm), on top of the decorticated bone and beneath the surgically resected infraspinatus tendon. Using this positioning the DERMAGRAFT was sandwiched between the bone and tendon. The DERMAGRAFT was held in place by mechanical forces due to the use of the sutures to reposition and tie down the detached tendon to the underlying bone. This route of administration was selected to allow the DERMAGRAFT to interface directly with both bone and tendon tissues. A minimally invasive technique could have alternatively been used.

Frequency and Duration of Administration

The DERMAGRAFT was placed only once at the time of the initial surgical procedure.

Dose Levels

To address the impact of increasing levels of DERMAGRAFT at the wound site, either a single layer or three layers of the matrix was used. The three layers were created by folding the DERMAGRAFT on top of itself. The single and multiple layers of DERMAGRAFT were trimmed to an appropriate size to fit over the footprint created on the head of the humerus due to decortication of the bone. These levels of DERMAGRAFT were selected to represent the doses that would be evaluated in a human clinical study.

Study Evaluations:

Body Weights

Body weights of the individual sheep were recorded prior to surgery and at the time of euthanasia.

Serum Generation

Blood was collected for the generation of serum using appropriate blood collection tubes. The serum samples generated were aliquoted into labeled cryovials and the samples were made available to evaluate for antibody formation by the sheep against the DERMAGRAFT matrix.

Collection of Non-Shoulder Tissues

Tissues from various organs were collected at the time of sacrifice and the tissues stored in fixative (10% formalin) for potential histological analysis at a later date.

DERMAGRAFT Thawing Protocol
1. The DERMAGRAFT (DG) product was kept frozen at −75±10° C. until ready to thaw.
2. A bath containing water at 34° C. to 37° C. was prepared.
3. A sterilized vessel containing sterile 70% isopropyl alcohol was prepared and kept at room temperature.
4. A sterilized vessel containing sterile saline was prepared and kept at room temperature.
5. The box containing the DERMAGRAFT product was removed from either the freezer or shipping box containing dry ice.
6. The cardboard box was torn open along its perforation.
7. The foil pouch was removed from the box.
8. The foil pouch was torn open by hand at the tear notch.
9. The clear bag containing DERMAGRAFT was removed
10. The clear bag was quickly and completely submerged in the 34° C. to 37° C. water bath. Approximately 2 minutes were allowed for thawing. The process was complete when no ice crystals were visible.
11. The thawing processes was not longer than 3 minutes.
12. While thawing, one of the peel-off labels was removed (containing the product lot number and expiration date) and placed on the surgical chart for the individual sheep.
13. The clear bag was removed from the water.
14. While wearing surgical gloves, the clear bag was quickly added to the vessel containing sterile 70% alcohol. The bag was held in the alcohol for 20-30 seconds.
15. The excess alcohol was allowed to drain from the bag.
16. The bag was transferred to the vessel containing sterile saline and held in the saline for 10-20 seconds.
17. The bag was removed from the saline and the top of the bag was wiped off with a sterile gauze to remove excess fluid from the area where the bag was to be cut open.
18. While holding the bag by the outer edges (note: the region containing the DERMAGRAFT mesh was not touched), the top of the bag was cut open above the cut line, using sterilized scissors.
19. The liquid contents were gently poured out of the bag, making sure to not allow the DERMAGRAFT to come out of the bag (note: if DERMAGRAFT had detached from the inner bag, the top of the bag would have been pinched before pouring out solution).
20. The bag was refilled with sterile room temperature saline, held for 5 seconds and then the saline was poured out.
21. The bag was refilled with sterile room temperature saline, held for 5 seconds and then the saline was poured out.
22. The bag was refilled with sterile room temperature saline, held for 5 seconds and then the saline was poured out.
23. The bag was filled for a fourth time with sterile saline and held until the time of use or the DERMAGRAFT was removed from the bag and placed in a vessel containing sterile room temperature saline. (The DERMAGRAFT may be held in the saline for up to 30 minutes before placement in the animal.)
24. The DERMAGRAFT was removed from the clear bag by placing the bag onto a sterile surface, and using sterilized scissors to cut around the edges of the bag to free the DERMAGRAFT. This included cutting off the “weld” sites where the DERMAGRAFT was directly held to the bag.
25. The top layer of the clear bag was carefully lifted away from the DERMAGRAFT and discarded.
26. With sterilized forceps, the DERMAGRAFT was lifted up and placed in a sterile petri dish containing sterile saline. (The DERMAGRAFT may be held in the saline for up to 30 minutes total between final rinse and before placement in the animal.)

### Example 10

### Histological and Biomechanical Assessment of a Rotator Cuff Bone-Tendon Interface

The biomechanical and histological responses of the reattached tendon following DERMAGRAFT treatment can be evaluated using the same sheep model as described in Example 9. It should be appreciated to those skilled in the art that the exact times listed in this Example of sample analysis, tissue harvesting, and assessment of product safety and efficacy, are not set and can be varied, with different times providing results that can be used to assess the healing capability of the test product.

The study design provides sufficient group sizes to allow for meaningful analysis of data. The number (n) of animals for biomechanical testing was calculated using coefficient of variance data from control animals of previously run studies, to allow for the detection of a 50% difference in mechanical strength between test and control groups.

TABLE 5
Study Design

| | | | Radiographic | | |
|---|---|---|---|---|---|
| | | | & Histological | | |
| | | | Assessment | | |
| | | | (n) | | BioMechanics |
| | Sheep # | | Week | Week | (n) |
| Group # | (n) | Treatment | 10⁽*⁾ | 20⁽*⁾ | Week 10 |
| 1 | 15⁽⁺⁾ | Suture only | 2 | 3 | 10 |
| 2 | 15⁽⁺⁾ | Suture + 1 layer of | 2 | 3 | 10 |
| | | DERMAGRAFT | | | |
| 3 | 15⁽⁺⁾ | Suture + 3 layers of | 2 | 3 | 10 |
| | | DERMAGRAFT | | | |

⁽*⁾The sheep that are sacrificed at the 10 (n = 2) and 20 (n = 3) week time points and are assigned to have the rotator cuffs analyzed by histology. Harvested shoulders are also radiographically assessed to detect new bone formation.
⁽⁺⁾All of the sheep have blood draws taken both pre-surgery and immediately prior to euthanasia. Blood draws are also taken at scheduled intervals (2, 4, 7, 14 and 28 days) for subgroups of three (3) animals from each study group. The blood drawn is used for: 1) the generation of serum for antibody analysis, and 2) for blood chemistry analysis.

The study uses 45 animals with one test site (the infraspinatus tendon of right shoulder) in each animal. The sheep are randomly divided into three groups of 15 (see Table 5). The surgically repaired rotator cuff tissues, from both DERMAGRAFT treated and control shoulders, are harvested and processed for the radiographic and histological assessment and for biomechanical testing. The shoulders scheduled for histological processing are first subjected to radiographic assessment to look for the presence of new bone formation. Assessments are made at the region adjacent to the head of the humerus where the tendon was detached, and at other sites away from the tendon attachment site for the presence of ectopic bone formation.

Animal Groupings

The 45 sheep are randomly assigned, prior to the initiation of the study, into the 3 treatment groups as shown in Table 5 herein. The order of the surgeries are randomized to ensure that the treatment groups are randomized across the multiple days that are required to complete the surgical procedures. The treatment received by each animal is recorded on the Operative Record sheet that is kept for each animal, individually.

Radiographs

At the time of the harvesting of the rotator cuff tissues for histological assessment, the proximal portion of the humerus and the attached tendon are assessed by radiography. Radiographs are taken using an axial approach of the bisected tendon/bone tissue to look for the formation of bone at the surgical site and in adjacent soft tissues. Additionally, radiographs are taken of bisected contra-lateral nonsurgically repaired sites as controls for comparison to the treated shoulders. The radiographs are reviewed by a qualified veterinary radiologist, blinded to the treatment group of each specimen.

Histological Assessment

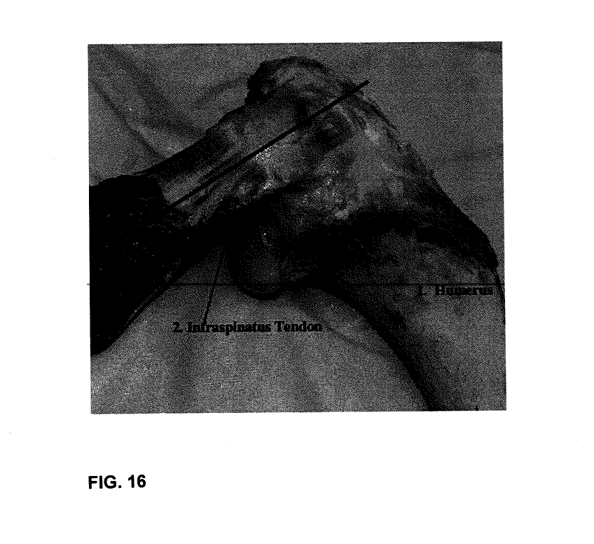

Following animal euthanasia at weeks 10 and 20, the operated (right) shoulders are harvested and delivered fresh for radiographic imaging and histological processing. Each shoulder is bisected through the infraspinatus and humeral attachment site into cranial and caudal halves using a scalpel for the tendon and a diamond blade saw for the humerus (Exakt Technologies, Oklahoma City, Okla.). To assure continuity in bi-sectioning the shoulder at the same location for each of the animals, the location for the bi-section is based upon the center bone tunnel that was created at the time of the initial surgical procedure. A photo of the bisection plane is shown in FIG. 16. Digital images are taken of each specimen during trimming and the two halves are subjected to radiographic imaging. At the time of bisection, a small piece of tissue approximately 2×2 mm is collected from the central portion of the bisected shoulder and subjected to PCR analysis.

The cranial and caudal aspects are processed for decalcified histology and embedded separately in paraffin (blocks are denoted as Block A “cranial” and Block B “caudal”). The specimens are initially fixed in 10% neutral buffered formalin for approximately 2 weeks. The specimens are then decalcified in formic acid+formaldehyde for approximately 2-3 weeks, as determined by x-ray for demonstrating complete decalcification. Once decalcified, the specimens are dehydrated using graded solutions of alcohol, cleared with xylene, and infiltrated with paraffin using a Sakura Tissue TEK V.I.P. Processor, Sakura Finetek USA, Inc., Torrance, Calif. The specimens are then embedded using standard paraffin histology techniques on a Shandon Histocentre 2, Thermo Shandon, Inc, Pittsburgh, Pa. The paraffin blocks are faced and approximately 5-8 micron thick sections cut on a Shandon Finesse rotary microtome (Thermo Shandon, Inc, Pittsburgh, Pa.).

Twelve histological sections are obtained from each shoulder, six from each block (A, B). Once the blocks are faced, 3 sections are cut from the medial side of the block. The blocks are then faced approximately 250 microns deeper into the tissue and another 3 sections are cut. At each 250 micron increment, three histological sections are cut; one section is stained with Hematoxylin-Eosin (H&E), one is stained with picrosirius-red, and one is stained with toluidine blue. The total number of slides generated is 120 (10 shoulders×12 slides per shoulder). Sections obtained are of the quality necessary for a pathological assessment.

High-resolution digital images are acquired by field for the entire stained slide using an Image Pro Imaging system (Media Cybernetics, Silver Spring, Md.) and a Nikon E800 microscope (AG Heinze, Lake Forest, Calif.), Spot digital camera (Diagnostic Instruments, Sterling, Heights, Mich.), and a Pentium IBM-based computer with expanded memory capabilities (Dell Computer Corp., Round Rock, Tex.). Once the slides have been imaged, they are subjected to blinded pathological evaluation.

BioMechanical Testing

Tissue Harvesting and Processing

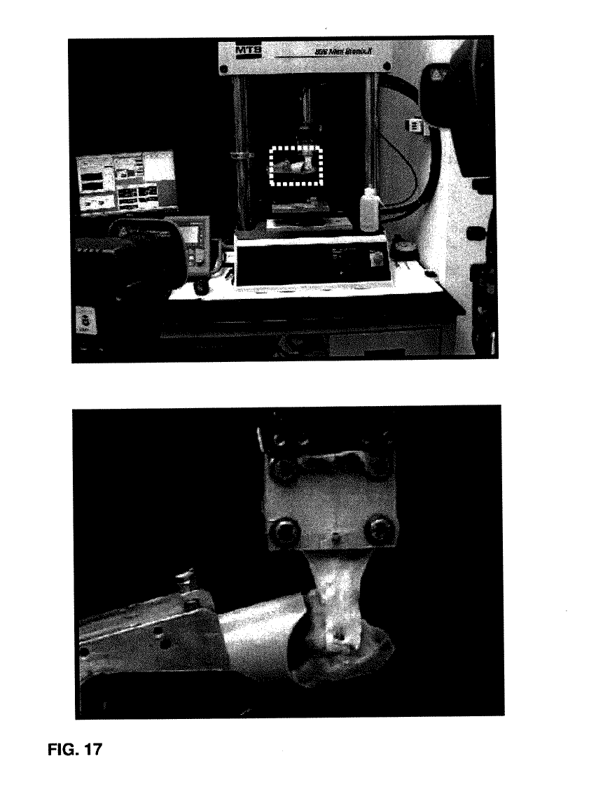

Shoulders from animals allocated for biomechanical testing are harvested and denuded of all soft tissues but leaving the humerus-infraspinatus tendon construct intact. A total of 40 shoulders are tested, ten (10) from each study group and ten (10) contra-lateral non-surgically repaired shoulders collected from the control group (Group 1). Specimens are wrapped in saline soaked gauze and stored at −20° C. until testing. The humeri are thawed on the day of analysis and then potted in 2″ PVC pipe using high strength Dynacast resin. Specimens are kept moist during the potting preparation and biomechanical testing with a saline spray at 15 minute intervals. The potted humeri are mounted in a custom-designed testing fixture that is rigidly attached to the materials testing system loading frame (FIG. 17) (MTS MiniBionix, Edan Prairie, Minn.; Fig SS). Each tendon is held in place using a cryo-clamp. Testing commences when the thermocouple attached to the cryo-clamp registers −20° C., which is a sufficient temperature to ensure secure and rigid coupling between the tendon and clamp.

Phase 1: 30 Cycle Dynamic Preconditioning

A cyclic loading test is initially employed to precondition the rotator cuff repair. A 10 Newton (N) preload is applied in force control for two minutes, following which the repaired construct is cyclically preconditioned in a force-control protocol from 10 to 50 N at 0.25 Hz for 60 cycles to reach a steady-state condition. Sixty (n=60) cycles was chosen based on pilot experiments demonstrating that the slope of the displacement versus time curve reaches a repeatable steady-state behavior between 50 and 60 cycles. Elongation and peak-to-peak displacement is determined during the cyclic preconditioning test. Elongation is defined as the distance in y-displacement between the 1st cyclic peak and the 60th cyclic peak. Peak-to-peak displacement is defined as the average of the local minimum to maximum of the 58th, 59th and 60th cycles.

Phase 2: Quasi-Static Failure Loading

Following preconditioning, the repaired constructs are loaded to failure under displacement control at a rate of 1 mm/s. Biomechanical parameters of interest include ultimate load-to-failure and quasi-statistic stiffness (defined as the slope of the load displacement curve). The failure mechanism is also documented for each specimen. Digital images are taken as appropriate. The quantitative measurements, including load to failure and stiffness, are analyzed for each test group and compared between groups and to the control non-surgically treated shoulders. A One-Way ANOVA followed by a Tukey's post-hoc multiple comparison test is used to identify significant differences in biomechanical parameters between treatment groups. Significance is set at p<0.05 and all analyses are performed with SigmaStat 3.1 (Systat Software, Inc., San Jose, Calif.).

Serum Antibody Analysis

To determine if the implantation of DERMAGRAFT into the sheep at the site of an acute rotator cuff injury leads to the formation of anti-DERMAGRAFT antibodies, serum samples are collected at multiple time points. Serum is collected from all sheep prior to the initial surgical procedure for the placement of DERMAGRAFT, or control surgery alone, depending upon the treatment group. Serum samples are also collected at multiple time points following the surgical procedure from all three study groups using subsets of sheep. Serum is also collected from all sheep at the time of euthanasia at either 10 or 20 weeks.

The serum samples are analyzed for the presence of antibodies directed against DERMAGRAFT. The methods used include the use of a solid-phase binding ELISA.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only and the invention is described in detail by the claims that follow.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of maintaining exogenous, viable fibroblasts between a detached tendon and a bone in a subject, the method comprising:
implanting an in vitro-prepared dermal tissue that comprises viable fibroblasts between a detached tendon and a bone in a subject.

2.-4. (canceled)

5. The method of claim 2, wherein the fibroblasts are dermal fibroblasts.

6.-13. (canceled)

14. The method of claim 1, wherein the in vitro-prepared dermal tissue further comprises a growth factor or cytokine selected from: PDGF-A, IGF, KGF, HBEGF, TGF-α, TGF-β1, TGF-β3, VEGF, G-CSF, IL-6, IL-8, Angiopoietin I, HGF and SPARC.

15.-16. (canceled)

17. The method of claim 1, wherein the in vitro-prepared dermal tissue is attached to a scaffold formed into a three-dimensional structure.

18.-20. (canceled)

21. The method of claim 17, wherein the scaffold is composed of a biodegradable material.

22. The method of claim 21, wherein the biodegradable material is polyglycolic acid, polylactic acid, or a co-polymer thereof.

23.-29. (canceled)

30. The method of claim 1, wherein the in vitro-prepared dermal tissue is a sheet.

31.-51. (canceled)

52. A method of delivering exogenous cytokines and/or growth factors to a damaged bone-tendon interface, the method comprising:
implanting an in vitro-prepared dermal tissue at the damaged bone-tendon interface in the subject.

53. The method of claim 52, wherein the in vitro-prepared dermal tissue comprises an exogenous growth factor or cytokine selected from: PDGF-A, IGF, KGF, HBEGF, TGF-α, TGF-β1, TGF-β3, VEGF, G-CSF, IL-6, IL-8, Angiopoietin I, HGF and SPARC.

54.-90. (canceled)

91. A method of promoting wound healing at a damaged bone-tendon interface in a subject, the method comprising:
implanting an in vitro-prepared dermal tissue at the damaged bone-tendon interface in the subject.

92. A method for implanting an in vitro-prepared tissue between a detached tendon and a bone in a subject, the method comprising:
attaching a first portion of at least one suture to a detached tendon and attaching a second portion of the at least one suture to a bone in a subject;
obtaining an implantable article of claim 110 comprising an in vitro-prepared tissue comprising an outer border, an inner region and a slit, the slit extending along a length from the outer border to the inner region; and
positioning the implantable article such that the at least one suture fits transversely within the slit.

93.-97. (canceled)

98. A method for implanting an in vitro-prepared tissue between a detached tendon and a bone in a subject, the method comprising:
attaching a first portion of at least one suture to a detached tendon and attaching a second portion of the at least one suture to a bone in a subject;
obtaining an implantable article of claim 167 comprising (a) an in vitro-prepared tissue comprising a first outer border, a first inner region and a first slit, the first slit extending along a length from the first outer border to the first inner region, and (b) a first support structure having a second outer border, a second inner region and a second slit, the second slit extending along a length from the second outer border to the second inner region, the in vitro-prepared tissue and first support structure being arranged such that the first slit is aligned with the second slit; and
positioning the implantable article such that the at least one suture fits transversely within the first slit and second slit.

99.-101. (canceled)

102. A method for implanting an in vitro-prepared tissue between a detached tendon and a bone in a subject, the method comprising:
attaching a first portion of at least one suture to a detached tendon and attaching a second portion of the at least one suture to a bone in a subject;
obtaining an implantable article of claim 203 comprising (a) an in vitro-prepared tissue comprising a first outer border, a first inner region and a first slit, the first slit extending along a length from the first outer border to the first inner region, (b) a first support structure having a second outer border, a second inner region and a second slit, the second slit extending along a length from the second outer border to the second inner region, and (c) a second support structure comprising a third outer border, a third inner region and a third slit, the third slit extending along a length from the third outer border to the third inner region, wherein the first support structure is attached to the second support structure such that the in vitro-prepared tissue is immobilized between the first support structure and the second support structure and the first slit, second slit and third slit are all aligned; and
positioning the implantable article such that the at least one suture fits transversely within the first slit, second slit and third slit.

103.-106. (canceled)

107. A method for implanting an in vitro-prepared tissue between a detached ligament and a bone in a subject, the method comprising:
attaching a first portion of at least one suture to a detached ligament and attaching a second portion of the at least one suture to a bone,
obtaining an implantable article of claim 110 comprising (a) an in vitro-prepared tissue comprising an outer border, an inner region and a slit, the slit extending along a length from the outer border to the inner region, and
positioning the implantable article such that the at least one suture fits transversely within the slit.

108. (canceled)

109. A method for implanting an in vitro-prepared tissue between a detached tendon and a bone in a subject, the method comprising:
attaching a first portion of at least one suture to a detached tendon and attaching a second portion of the at least one suture to a bone in a subject; obtaining an implantable article comprising (a) an in vitro-prepared tissue comprising a first outer border, a first inner region and a first slit, the first slit extending along a length from the first outer border to the first inner region, and (b) a first support structure having a second outer border, a second inner region and a second slit, the second slit extending along a length from the second outer border to the second inner region, the in vitro-prepared tissue and first support structure being arranged such that the first slit is aligned with the second slit; and positioning the implantable article such that the at least one suture fits transversely within the first slit and second slit, wherein the in vitro-prepared tissue comprises fibroblasts.

110. An implantable article comprising:
an in vitro-prepared tissue comprising an outer border, an inner region and a slit, the slit extending along a length from the outer border to the inner region.

111.-149. (canceled)

150. A cutting article comprising:
a solid support having a cutting edge, the cutting edge delineating an outer border and a slit, the slit being delineated by a first portion of the cutting edge extending inwardly along a length from the outer border and a second portion of the cutting edge extending inwardly along a length from the outer border, the first portion and second portion of the cutting edge being joined at an inner region.

151.-161. (canceled)

162. A cutting article comprising
a solid support having a plurality of non-overlapping cutting edges, each cutting edge delineates an outer border and a slit, the slit being delineated by a first portion of the cutting edge extending inwardly along a length from the outer border and a second portion of the cutting edge extending inwardly along a length from the outer border, the first and second portions of the cutting edge being joined at an inner region.

163. A method for preparing an implantable article comprising:
obtaining an in vitro-prepared tissue, and
cutting the in vitro-prepared tissue to form an outer border and a slit, the slit extending along a length from the outer border to an inner region of the in vitro-prepared tissue.

164. A method for preparing an implantable article comprising:
obtaining a scaffold,
cutting the scaffold to form an outer border and a slit, the slit extending along a length from the outer border to an inner region of the in vitro-prepared tissue, and
culturing mammalian cells on the scaffold under conditions that permit attachment of the mammalian cells to the scaffold and synthesis of extracellular matrix by the mammalian cells.

165.-166. (canceled)

167. An implantable article comprising:
an in vitro-prepared tissue comprising a first outer border, a first inner region and a first slit, the first slit extending along a length from the first outer border to the first inner region; and
a first support structure having a second outer border, a second inner region and a second slit, the second slit extending along a length from the second outer border to the second inner region, wherein the in vitro-prepared tissue is positioned adjacent to the first support structure such that the first slit is aligned with the second slit.

168.-200. (canceled)

201. The implantable article of claim 167, further comprising a second support structure comprising a third outer border, a third inner region and a third slit, the third slit extending along a length from the third outer border to the third inner region,
wherein the first support structure is attached to the second support structure such that the in vitro-prepared tissue is immobilized between the first support structure and the second support structure and the first slit, second slit and third slit are all aligned.

202.-217. (canceled)

218. A device for introducing an implantable article to a subject, the device comprising:
an elongated sheath having an distal opening, a distal internal cavity and a proximal internal cavity, the distal internal cavity being shaped to receive an implantable article through the distal opening,
a plunger having a distal end and a proximal end, the distal end being adapted to interface with the implantable article in the distal internal cavity, the proximal end being movably fitted within the proximal internal cavity, and
an actuator for moving the plunger axially within the sheath between a retracted position and an extended position, the actuator being coupled with the plunger at the proximal end,
wherein movement of the plunger to the retracted position permits the implantable article to be received in the distal internal cavity, and movement of the plunger to the extended position causes the implantable article to be ejected from the distal internal cavity.

219.-222. (canceled)

223. A device for introducing an implantable article to a subject, the device comprising:
an elongated sheath means having a distal internal cavity and a proximal internal cavity, the distal internal cavity being shaped to receive an implantable article through the distal opening,
a plunger means having a distal end and a proximal end, the distal end being adapted to interface with the implantable article, the proximal end being movably fitted within the proximal internal cavity, and
an actuator means for moving the plunger axially within the sheath between a retracted position and an extended position, the actuator being coupled at the proximal end of the plunger,
wherein movement of the plunger to the retracted position permits the implantable article to be received in the distal internal cavity, and movement of the plunger to the extended position causes the implantable article to be ejected from the distal internal cavity.

224.-225. (canceled)

226. An implantable article comprising:
an in vitro-prepared tissue comprising an outer border, an inner region and a slit, the slit extending along a length from the outer border to the inner region, wherein the in vitro-prepared tissue comprises fibroblasts.


SEQUENCE LISTING (6 sequences)

SEQ ID NO: 1 - DNA, 20 bp, Artificial Sequence
  Synthetic Oligonucleotide
agtgtgaaacgggagaaaac

SEQ ID NO: 2 - DNA, 20 bp, Artificial Sequence
  Synthetic Oligonucleotide
tacaacctgttgtccagttg

SEQ ID NO: 3 - DNA, 20 bp, Artificial Sequence
  Synthetic Oligonucleotide
aggcaacgtccaggatagag

SEQ ID NO: 4 - DNA, 21 bp, Artificial Sequence
  Synthetic Oligonucleotide
gcaattcttcggcagcatctt

SEQ ID NO: 5 - DNA, 21 bp, Artificial Sequence
  Synthetic Oligonucleotide
cgcagacaggatgcagaaaga

SEQ ID NO: 6 - DNA, 21 bp, Artificial Sequence
  Synthetic Oligonucleotide
gctgatccacatctgctggaa